(19) United States
(12) Patent Application Publication
(10) Pub. No.: US 2017/0202789 A1
(43) Pub. Date: Jul. 20, 2017

(54) METHODS AND RELATED COMPOSITIONS FOR IMPROVED DRUG BIOAVAILABILITY AND DISEASE TREATMENT

(71) Applicants: Frederick A. Sexton, Rumson, NJ (US); Timothy S. Tracy, Lexington, KY (US); Sitaraman Krishnan, Potsdam, NY (US); Vankat K. Vendra, Louisville, KY (US); James M. Myrick, Postdam, NY (US)

(72) Inventors: Frederick A. Sexton, Rumson, NJ (US); Timothy S. Tracy, Lexington, KY (US); Sitaraman Krishnan, Potsdam, NY (US); Vankat K. Vendra, Louisville, KY (US); James M. Myrick, Postdam, NY (US)

(73) Assignee: NEW WORLD PHARMACEUTICALS, LLC, Charleston, SC (US)

(21) Appl. No.: 15/326,016

(22) PCT Filed: Jul. 16, 2015
(86) PCT No.: PCT/US2015/040811

§ 371 Date: Jan. 12, 2017

Related U.S. Application Data
(60) Provisional application No. 62/025,429, filed on Jul. 16, 2014.

(51) Int. Cl. A61K 31/155; A61K 31/198; A61K 31/22; A61K 9/16; A61K 31/519; A61K 31/522; A61K 31/7072; A61K 31/7004; A61K 31/4164

(52) U.S. Cl. CPC A61K 31/155; A61K 31/7004; A61K 31/198; A61K 31/22; A61K 31/4164; A61K 31/519; A61K 31/522; A61K 31/7072; A61K 9/1652

(57) ABSTRACT

The present invention relates to methods and compositions for improved drug bioavailability and disease treatment, including treatment of diseases related to hormone modulation or CNS function. In certain embodiments, the instant invention provides methods for hormone modulation or improving CNS function, comprising administering to a subject in need thereof a composition comprising one or more hydrogel particles, wherein the one or more hydrogel particles are non-toxic and incorporate at least one active agent, wherein the one or more hydrogel particles release the active agent in a time-controlled and sustained manner in vivo.

### CROSS-REFERENCE TO RELATED APPLICATION

This application claims the benefit of priority of U.S. Provisional Application Ser. No. 62/025,429, filed Jul. 16, 2014. The foregoing application is incorporated herein by reference in its entirety.

### FIELD OF THE INVENTION

The present invention relates to methods and compositions for improved drug bioavailability and disease treatment.

### BACKGROUND OF THE INVENTION

Athletes must replenish and maintain nutrients during exercise for optimal performance. Particularly during longer periods of exercise, it is important to take in nutrients beyond just simple intake of water to replenish energy stores utilized during the athletic event. Numerous different foodstuffs have been tested for their ability to provide energy supplementation during exercise, including carbohydrates, protein, fats, and ergogenic substances.

The foodstuff most often evaluated for its ability to provide effective supplementation during athletic performance has been carbohydrates and the positive effects of carbohydrate supplementation on exercise performance have clearly been documented (1-3). Carbohydrates, stored as glycogen, are the major endogenous source of fuel for the body as they contain sugars, such as glucose and fructose. Glucose is particularly advantageous in that it is directly converted to energy with no lag, whereas fructose, other sugars, fats, and proteins require additional processing. Protein and fats have also been evaluated but, as described below, with less positive effects on athletic performance. Additionally, ergogenic substances, such as caffeine, have the ability to increase energy utilization but do not provide replenishment of spent energy sources.

Administration of beverages containing caffeine, B-vitamins, and amino acids has been promoted as increasing energy. Though some positive effects on performance have been noted with caffeine, it simply increases the body's use of current energy stores and does not provide new sources of energy or replenishment. No replicable effects of the other agents (B-vitamins or amino acids) have been noted.

In sum, it is well established that maintaining adequate available energy is key to maximum performance for both muscles and the brain to maintain activity, as well as mental focus.

Citation or identification of any document in this application is not an admission that such document is available as prior art to the present invention.

### SUMMARY OF THE INVENTION

The instant invention relates to a method of improving cognitive function, comprising administering to a subject in need thereof a composition comprising one or more hydrogel particles, wherein the one or more hydrogel particles (a) are non-toxic; and (b) incorporate at least one active agent, wherein the one or more hydrogel particles release the active agent in a time-controlled and sustained manner in vivo, wherein the administration of the composition improves cognitive function in the subject. In some embodiments, the improvements in cognitive function include improvements in attention, psychomotor, and/or memory abilities.

In some embodiments, the invention relates to a method of treating a central nervous system (CNS) disease or condition, comprising administering to a subject in need thereof a composition comprising one or more hydrogel particles, wherein the one or more hydrogel particles (a) are non-toxic; and (b) incorporate at least one active agent, wherein the one or more hydrogel particles release the active agent in a time-controlled and sustained manner in vivo, wherein the administration of the composition improves brain and/or spinal cord function in the subject. In some embodiments, the active agent is levodopa or phenylalanine. Examples of CNS diseases or conditions that may be treated include ischemia, a neurodegenerative disorder, a mental health disorder, a pain disorder, an addiction disorder, a brain or spinal cord injury, and a brain or spinal cord tumor.

In certain embodiments, the invention relates to a method of treating a metabolic disorder, comprising administering to a subject in need thereof a composition comprising one or more hydrogel particles, wherein the one or more hydrogel particles (a) are non-toxic; and (b) incorporate at least one active agent, wherein the one or more hydrogel particles release the active agent in a time-controlled and sustained manner in vivo, wherein the administration of the composition improves metabolic function in the subject. In some embodiments, the active agent is metformin. In further embodiments, the metabolic disorder is selected from the group consisting of: obesity, metabolic syndrome, and hypoglycemia. In other embodiments, the metabolic disorder is selected from the group consisting of diabetes, insulin resistance, hyperglycemia, and impaired glucose tolerance. In particular embodiments, the diabetes is selected from the group consisting of: type 1 diabetes, type 2 diabetes, gestational diabetes, and MODY (maturity onset diabetes of the young) diabetes. In a particular embodiment, the metabolic disorder is type 2 diabetes and the active agent is metformin.

In yet other embodiments, the invention relates to a method of increasing satiety hormone release, comprising administering to a subject in need thereof a composition comprising one or more hydrogel particles, wherein the one or more hydrogel particles (a) are non-toxic; and (b) incorporate at least one active agent, wherein the one or more hydrogel particles release the active agent in a time-controlled and sustained manner in vivo, wherein the administration of the composition increases satiety hormone release in the subject. In certain embodiments, the satiety hormone is selected from cholecystokinin (CCK), peptide YY (PYY), pancreatic polypeptide (PP), insulin, and incretins. In particular embodiments, the incretin is selected from the group consisting of: glucagon-like peptide 1 (GLP-1), oxyntomodulin, and glucose-dependent insulinotropic polypeptide.

In some embodiments, the invention relates to a method of decreasing hunger hormone release, comprising administering to a subject in need thereof a composition comprising one or more hydrogel particles, wherein the one or more hydrogel particles (a) are non-toxic; and (b) incorporate at least one active agent, wherein the one or more hydrogel particles release the active agent in a time-controlled and sustained manner in vivo, wherein the administration of the composition decreases hunger hormone release in the subject. In a particular embodiment, the hunger hormone is ghrelin.

In certain embodiments, the at least one active agent is a carbohydrate. In further embodiments, the carbohydrate is selected from the group consisting of: monosaccharides, disaccharides, polysaccharides, and combinations thereof. In particular embodiments, the carbohydrate is selected from the group consisting of: glucose, fructose, galactose, sucrose, maltose, lactose, dextrose, trehalose, polydextrose, dextrins, maltodextrins, corn syrup solids, starch, and combinations thereof. In a certain embodiment, the carbohydrate is glucose. In a further embodiment, the glucose is released in distal portions of the small intestine after administration of the composition to the subject.

In some embodiments, the active agent improves neurotransmitter efficacy.

In yet other embodiments, the active agent increases brain glycogen stores.

In other embodiments, the invention relates to a method of treating a cardiovascular disorder, a digestive disorder, an immune disorder, a pulmonary disorder, a viral disease, or a cancer, comprising administering to a subject in need thereof a composition comprising one or more hydrogel particles, wherein the one or more hydrogel particles (a) are non-toxic; and (b) incorporate at least one active agent, wherein the one or more hydrogel particles release the active agent in a time-controlled and sustained manner in vivo, wherein the administration of the composition improves the cardiovascular, digestive, immune, and/or pulmonary function in the subject and/or treats the viral disease and/or cancer in the subject. In some embodiments, the active agent is selected from the group consisting of: pravastatin, cimetidine, methotrexate, theophylline, and zidovudine.

In certain embodiments, the one or more hydrogel particles comprise one or more compounds that are temperature-sensitive. In some embodiments, the one or more compounds have a lower critical solution temperature in aqueous solution.

In certain embodiments, the one or more hydrogel particles comprise one or more compounds that are pH-sensitive. In some embodiments, the one or more compounds do not swell at pH 1-3.

In some embodiments, the one or more hydrogel particles comprise one or more compounds that are both temperature-sensitive and pH-sensitive. In some embodiments, the one or more compounds do not swell at pH 1-3.

In certain embodiments, the one or more hydrogel particles comprise one or more compounds that are crosslinked.

In some embodiments, the one or more hydrogel particles have a diameter between about 1 nanometer to about 1000 micrometers.

In some embodiments, the bioavailability of the active agent is improved (e.g., increased) by administration to a subject in need thereof according to a method of the instant invention.

### BRIEF DESCRIPTION OF THE DRAWINGS

FIG. 1 is a schematic depicting the metabolic steps converting glucose to energy.

FIG. 2 is a schematic depicting the metabolic steps converting fructose to energy.

FIG. 3 is a schematic depicting the metabolic steps converting galactose to energy.

FIG. 4 depicts SGLT1 and GLUT2 transporters in the cell.

FIG. 5 depicts a metformin hydrochloride (MH) calibration curve.

FIG. 6 depicts the release kinetics of metformin hydrochloride (MH) using a horizontal static diffusion cell. The release kinetics of MH from hydroxypropyl cellulose (HPC) particles as described herein was investigated. The control experiments were performed with 100 mg/mL MH solution at 37° C. with phosphate buffered saline (PBS) as the receptor medium. The standard HPC particle suspension saturated with MH provides a delay in the release of MH over an eight hour period.

FIG. 7 depicts laser diffraction analysis of particles formed by a temperature-induced precipitation crosslinking of HPC and CMC with TSTMP method as described herein.

### DETAILED DESCRIPTION OF THE INVENTION

As important as which foodstuffs can provide effective supplementation during athletic performance, is the composition of these foodstuffs and their method of delivery. For example, different forms of carbohydrates are converted into energy at different rates and may have different uptake properties. The optimal balance of energy source/form and delivery vehicle and rate has the potential to provide the greatest impact on the athletic performance and thus, athletic success.

### Forms of Energy Used by the Body

### Carbohydrates

Carbohydrates are the major source of fuel for the body (4), stored as glycogen. Because carbohydrates contain sugars, such as glucose and fructose, they provide a source of energy. Carbohydrates are classified as mono-, di-, and poly-saccharides based on the number of sugars contained in the molecule. Monosaccharides are the most readily available form of energy from carbohydrates since they require no processing prior to use. However, enzymes in the body can break down di- and polysaccharides to simple sugars for the provision of energy. The mono- and di-saccharide forms of carbohydrates are loosely categorized as simple carbohydrates, whereas polysaccharides are frequently classified as complex carbohydrates. Carbohydrates are the most commonly used exogenous energy source for the replenishment of nutrients during athletic performances, providing about 4 kcal/gm of energy. Because mono- and di-saccharides do not require extensive “processing” or breakdown to glucose or other simple sugars, they provide the most immediate and readily available energy source. Complex carbohydrates, such as polysaccharides will require more extensive processing within the intestinal tract to release the simple sugars and thus, do not provide as immediate a source of energy into the bloodstream. However, this can also be advantageous in that complex carbohydrates have been purported to provide a more sustained release of energy into the bloodstream (though recent work with maltodextrins suggests this is not universally true).

### Protein

Proteins are also an essential nutrient for growth and development, forming the building blocks for muscle and tissue. They are formed from chains of amino acids linked by peptide bonds. Because proteins are used to form muscle and tissue, they are important to an athlete's development and training. However, they are not as readily available as an energy source from endogenous pools and are usually the third accessed source of energy, utilized when carbohydrate and fat sources are low. However, protein does provide roughly the same amount of energy, ˜4 kcal/gm, as carbohydrates.

### Fats

Fats are also an important component of the diet and are consumed in the form of both saturated and unsaturated fats. They are stored in the body in either triglycerides or fatty acid form and then may be released following lipolysis, serving as a source of energy. Though fats are important for normal body function (both structurally and metabolically), they are not typically used as a foodstuff for nutrition during athletic performances, even though fats provide the highest amount of energy, ˜9 kcal/gm, of the three energy forms.

### Storage of Energy From Carbohydrates

### Carbohydrates

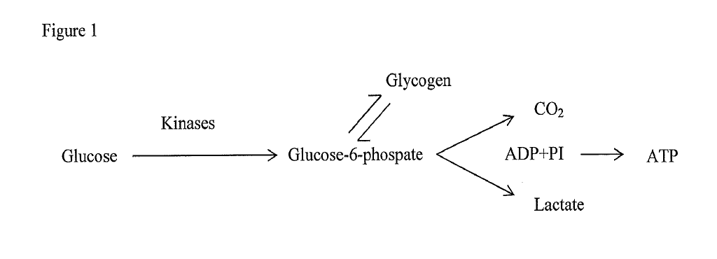

As stated above, carbohydrates can exist in polymeric chains and these polymeric polysaccharide chains are the form in which carbohydrates not used for immediate energy are stored. These polysaccharides, stored in the form of glycogen, are then available for breakdown to simple sugars (energy) during times when energy needs exceed exogenous energy consumption. By varying the types of sugars incorporated (e.g., glucose vs. fructose vs. galactose, etc.) the amount of energy provided and the ease of breakdown of the carbohydrate can be varied to achieve different levels of energy provision. Of the sugars, glucose is most readily converted to energy. Once glucose crosses the cell membrane, it is phosphorylated by glucokinase to form glucose-6-phosphate, the form in which glucose is stored within cells, such as the liver (FIG. 1). It should be noted that glucose-6-phosphate cannot cross the membrane and back into the blood in this form and must be cleaved back to glucose by a phosphatase before it can be transported back into the bloodstream. This helps serve as a storage mechanism, particularly within the liver, that still permits a quickly available source of energy. The glucose-6-phosphate that is stored within the cells can then be converted to ATP (FIG. 1). It is this ATP energy that is required for muscle contraction and brain action potential firing. Readily available endogenous energy stores can become depleted during strenuous and/or prolonged exercise, necessitating provision of exogenous nutrients in the form of carbohydrates. Providing energy supplementation during exercise can forestall the need to initiate glycogenolysis and draw from endogenous energy stores. Complementary to this, pre-loading of carbohydrates can serve to build up glycogen stores, which can be drawn upon to produce glucose. As during glycolysis, during glycogenolysis, pyruvate is a by-product that can bind to the protons produced during the breakdown of glucose and provide a buffering to reduce acidosis and the typical “muscle burn.” See, for example, Kravitz, L. (2005) “Lactate: Not guilty as charged” IDEA Fitness Journal 2 (6):23-25.

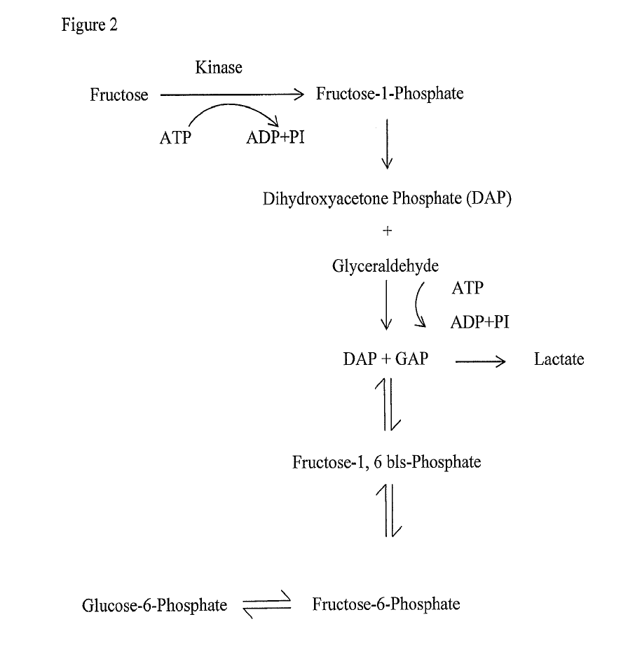

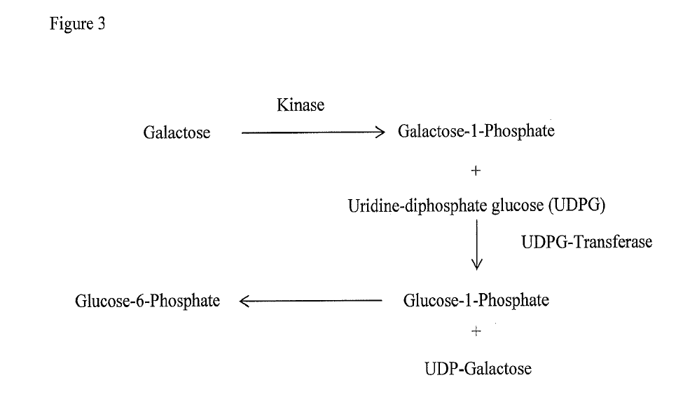

Fructose and galactose are also sources of energy but require additional metabolic steps, as opposed to glucose. They are phosphorylated by fructokinase and galactokinase, respectively (FIGS. 2 and 3). It should be noted that hexokinase can phosphorylate all six-member ring sugars and does so at a much lower Michaelis constant, Kₘ, than any of the kinases listed above. (Kₘ in the Michaelis-Menten rate equation is the substrate concentration at which the rate of the enzymatic reaction is half the maximum rate). However, hexokinase is readily subject to product inhibition and thus, has a low capacity due to this feedback inhibition. Fructose is frequently added to energy drinks and supplements as it is somewhat sweeter at room temperature and can improve the palatability of the drink supplement. Though the room temperature form of fructose (a 5-member furanose) is sweeter than glucose, the 6-member pyranose form that exists at higher temperatures (e.g., during cooking) is no sweeter than glucose. Fructose administration results in a lesser increase in plasma insulin levels than glucose and also reduces lipolysis to a smaller extent (5). However, fructose also undergoes a lower rate of oxidation than glucose. Work from Jandrain et al., (6), using a 13C labeling technique, has demonstrated that fructose consumed during exercise is oxidized at a slower rate than glucose and its availability as an energy source is also less than that of glucose. As a result, significantly less glucose is produced (i.e., the conversion of fructose into glucose) when fructose is the energy source as compared to glucose. In addition, fructose usage by muscles is limited since the only kinase in muscle that phosphorylates either glucose or fructose is hexokinase and hexokinase has a strong preference for glucose as a substrate (7). Glucose present at the muscle competes with fructose for phosphorylation, resulting in less fructose being converted to energy within muscle cells. Thus, fructose can serve as an energy source in energy supplementation products and at room temperature can provide more sweetening than glucose, but undergoes oxidation at a slower rate and is less available for oxidation.

Galactose is absorbed through the intestine by the same transporters that transport glucose but requires different transporters (than glucose) to enter the liver. Similar to fructose, galactose also exhibits a slower oxidation rate (8). Galactose is metabolized in the cells to galactose-1-phosphate and requires a phosphoglucomutase enzyme to convert it to glucose-6-phosphate where it can then enter the normal glycolytic pathway (FIG. 3). It is these additional steps and potential differences in rate of absorption that make galactose slightly slower in the provision of energy.

Other carbohydrates that have been included in energy supplementation products include maltose and maltodextrins (glucose polymers). Maltose appears to be oxidized at rates similar to glucose (9) and is likely absorbed at the same rate as glucose, as well. Maltodextrins have been frequently used as a carbohydrate source in energy supplementation drinks due to their relatively low osmolality and for their lack of any noticeable taste characteristics. The use of maltodextrins in energy supplementation products has been associated with similar oxidation rates as those of glucose and their rate of absorption (i.e., delivery of carbohydrate) into the intestine was also similar to that of glucose (10). This finding of comparability to glucose is particularly interesting since it implies that the rate of breakage of the polymer bonds is not the rate-limiting step in delivery and oxidation. This calls into question claims that the use of maltodextrins in energy supplements are different than directly providing sugars (e.g., glucose and fructose) and that they (maltodextrins) might provide a sustained energy source. Thus, simply providing complex carbohydrates, such as maltodextrins, may not effectively provide sustained energy.

### Absorption of Glucose

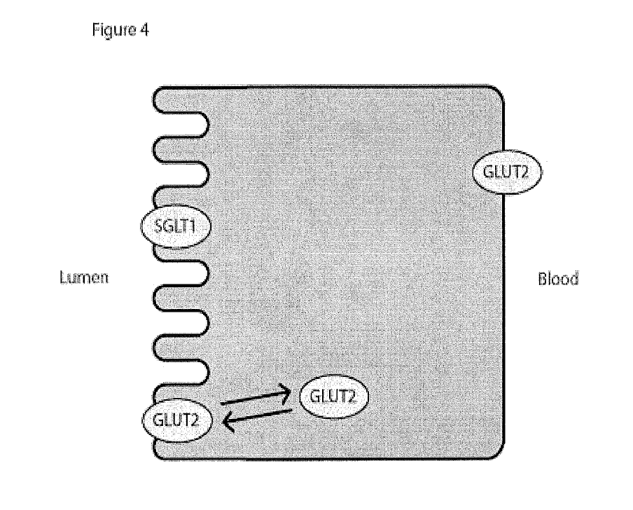

Glucose, from carbohydrates, is absorbed through the small intestinal wall by the SGLT1 and GLUT2 transporters for transfer to the bloodstream (FIG. 4). SGLT1 is a high affinity/low capacity glucose transporter present in the small intestine. At low gut glucose concentrations, the uptake of glucose is carried out predominantly by the SGLT1 transporter, facilitated by the high affinity nature of this transporter in helping assure glucose uptake. However, the SGLT1 transporter is also easily saturated and thus, is not able to provide sufficient capacity for glucose uptake in the presence of high gut glucose concentrations. Therefore, in the presence of high gut glucose concentrations the GLUT2 transporter is recruited to the apical membrane of the intestinal epithelium, where it serves as a low affinity/high capacity glucose transporter. Together, these two transporters work in a complementary fashion to modulate glucose uptake in the small intestine. In certain embodiments, the inventive methods described herein exploit the affinity, uptake and saturation characteristics of these transporters through release and delivery methods of simple and complex carbohydrates to affect blood glucose concentrations in a predictable manner. For example, in certain embodiments, the instant methods relate to delivery and release methods that engage each of the two transporters (SGLT1 and GLUT2) in a systematic and sustained manner For example, in certain embodiments, the inventive methods described herein involve a delivery system that maintains the released glucose in close proximity to the transporters and thus, results in a continuous supply to the transporter, maximizing glucose absorption. In certain embodiments, the methods of the instant application produce both immediate increase in blood glucose levels within a desired range and a more efficient overall uptake of glucose from the carbohydrate source. In some embodiments, use of such a formulation reduces the need for multiple “feedings” that may result in gastrointestinal effects and allows for alternating intake of pure water for strict fluid replacement. This ability to produce both immediate increases in blood glucose and more efficient uptake of glucose from carbohydrate energy supplementation products would not only benefit athletes in standard duration competitions (e.g., up to 2 hours) but would be particularly beneficial for prolonged athletic competitions such as a marathon or ultra-endurance competitions that require multiple feedings. In addition, energy replacement formulations that provide for more efficient and effective uptake of glucose would be expected to produce more effective replenishment of the glycogen reserve post-exercise and thus, improve recovery. In embodiments where a treatment method of the invention uses a formulation that delivers a reasonable volume of energy supplementation that does not result in gastrointestinal (GI) distress yet produces a delivery of glucose that maintains contact with glucose transporters in a fashion that results in optimal glucose uptake through constant saturation of the transporters over an extended time, improved output potential (e.g., athletic performance) and recovery via enhanced glycogen replenishment should be expected.

With an increase in blood glucose comes a concomitant increase in insulin levels. This increase in insulin levels serves to help move the glucose into the cells for processing and provision of energy. However, too large a spike in glucose can result in a substantial rise in insulin levels and thus, an overall reduction in blood glucose (i.e., hypoglycemia leading to a “crash” in energy). Thus, in certain embodiments, it is desirable to “tune” the release of glucose into the system to provide immediate energy needs but not more than needed since the rise in insulin may limit the longer-term effect of this rise in glucose. In particular embodiments, systems that produce necessary but not excessive rises in glucose immediately and an “extended” release of glucose to maintain glucose concentrations would be most ideal.

It has also been demonstrated that the uptake of glucose varies throughout the length of the small intestine (11). In a clinical study that infused glucose over either the first 60 cm or greater than the first 60 cm of the small intestine, investigators observed that the rise in plasma glucose and insulin was greatest in the infusion over the segment beyond 60 cm. Accordingly, in some embodiments, the methods of the instant invention provide glucose delivery systems that release glucose in the more distal segments of the small intestine, resulting in greater rises in blood glucose and thus, better replenishment of energy stores. This has important implications for energy replacement strategies. In certain embodiments, the use of formulations that delay release of glucose to the more distal segments of the small intestine produce greater rises in blood glucose and thus, more effective and efficient energy replacement.

Interestingly, this same study (11) also observed significant findings with regard to levels of satiety and hunger hormones and the location of glucose uptake in the small intestine. Though there are several satiety hormones, cholecystokinin (CCK) and glucagon-like peptide 1 (GLP-1) appear to be two of the more prominent satiety hormones and ghrelin is a prominent hunger hormone (12). Little and colleagues (11) observed that when glucose was infused in the more distal segments, not only was the rise in plasma glucose higher than during infusion in the proximal segments but also, levels of the satiety hormone GLP-1 was higher, as well. Furthermore, levels of the “hunger” hormone ghrelin were decreased to a greater extent following distal segment glucose infusion.

### Physiological Considerations of Energy Delivery Through the GI Tract

When considering delivery of “energy” in the form of carbohydrates to the gastrointestinal tract during exercise, one must also take into account changes in blood flow in the GI tract that occur as a mechanism to shunt blood from the gut to skeletal muscles to address the increased energy needs of the muscle tissues. It has been estimated that during exercise, blood flow in the GI tract may decrease as much as 70% (13), in an attempt to provide oxygen and nutrients to high consumption tissues, such as muscle (Table 1).

TABLE 1
Blood Flow Distribution During Rest and Exercise

| | Rest | | Heavy Exercise | |
|---|---|---|---|---|
| | | % of total | | % of total |
| | | cardiac | | cardiac |
| | mL/min | output | mL/min | output |
| Splanchnic (gastric, small | 1.4 | 24 | 0.3 | 1 |
| intestinal, colonic, pancreatic, | | | | |
| hepatic and splenic) | | | | |
| Renal | 1.1 | 19 | 0.9 | 4 |
| Brain | 0.75 | 13 | 0.75 | 4 |
| Coronary | 0.25 | 4 | 1 | 4 |
| Skeletal muscle | 1.2 | 21 | 22 | 86 |
| Skin | 0.5 | 9 | 0.6 | 2 |
| Other | 0.6 | 10 | 0.1 | 0.5 |
| Total Cardiac Output | 5.8 | 100 | 25.65 | 100 |

From: Parks DA and Jacobson ED. Physiology of the splanchnic circulation. Arch Intern Med 1985; 145: 1278-1281.

Conversely, provision of glucose in the GI tract may increase blood flow by as much as 40% (14), presumably to help increase absorption of this important nutrient and energy source. Thus, there is an overall net reduction in blood flow in the GI tract during exercise, even when consuming a glucose-containing product. This has implications for uptake and absorption of glucose during energy replacement in that less uptake and reduced metabolism of glucose may occur during exercise. Products that overcome this situation and maximize absorption of glucose would exhibit the most beneficial effect, for example, products that maximize contact time of glucose molecules with absorptive transporters, such as the SGLT1 and GLUT2 transporters that serve to move glucose molecules across cell membranes and into the circulation.

Glucose, from carbohydrates, is absorbed through the small intestinal wall by the SGLT1 and GLUT2 transporters for transfer to the bloodstream and eventual conversion to ATP. SGLT1 is a high affinity/low capacity glucose transporter that is quickly saturated. However, GLUT2, which in the presence of high glucose concentrations is recruited to the apical membrane of the small intestine, is a low affinity/high capacity glucose transporter and together, these two transporters modulate glucose uptake. In certain embodiments, the inventive methods described herein exploit these transporter characteristics by regulating the rate of carbohydrate (e.g., glucose) delivery to the small intestine, producing both an immediate increase in blood glucose levels and a sustained level of blood glucose, which, in particular embodiments, may be beneficial for prolonged athletic competitions.

In addition, studies have demonstrated that GI motility is also reduced during exercise, again, without being bound to theory, presumably to reduce energy usage in non-skeletal muscle tissues and facilitate greater energy usage in muscles used for the activity. This may have implications for the rate of delivery of glucose from energy supplementation products. Because of this reduction in motility, immediate release products may “dump” significant glucose into the body in a short period of time. Though beneficial in some instances in the short term, additional feedings may be needed which could result in fullness and GI upset due to the volume being retained higher in the GI tract.

### Use of Energy During Athletic Performance

The body can utilize either aerobic or anaerobic pathways to convert nutrients to energy during exercise. The reliance on either or both of these pathways to provide energy during exercise is dependent on both the duration and intensity of the exercise.

The body is not capable of storing a large amount of ATP, the energy source for muscles (15). However, through the ATP-creatine phosphate anaerobic energy pathway, about 10 seconds worth of energy is available for use in short bouts of exercise (e.g., a 100-meter sprint). The muscles are able to store about 2-3 seconds worth of ATP for use as an energy source that is used for these short duration, high intensity activities. Providing additional energy that fuels another 6-8 seconds of activity, the body is able to rapidly convert creatine phosphate to ATP. Once these two energy sources are depleted the body then will have to convert to alternative pathways to produce energy.

For those activities lasting more than about 10 seconds, the body must utilize anaerobic and/or aerobic energy pathways depending on the duration and intensity of the activity. Glycolysis is an anaerobic energy pathway that breaks down glucose-6-phosphate to produce ATP, with lactate being a by-product of this reaction (FIG. 1). This process does not require oxygen to cause the partial breakdown of glucose. The anaerobic glycolysis pathway is most useful in producing energy for short duration, high intensity activities that last only a few minutes. Though not as rapidly acting as the ATP-creatine phosphate pathway in providing energy, glycolysis is a reasonably rapid energy source for ATP production. Because it does not require the circulatory system to deliver more oxygen to the tissues, it is relatively effective for these types of short duration, high intensity activities such as a 1500 meter run. However, the consequence of the activation of this biochemical pathway is the build up of lactic acid that occurs and can result in muscle pain, burning and fatigue. This build up of lactic acid prevents maintaining this level of high intensity for prolonged periods of time.

Because many types of athletic performances are carried out for more prolonged periods of time, such as an extended match of tennis or soccer, a marathon run or a triathlon, aerobic metabolism must be engaged to provide energy for these activities of longer duration. Aerobic metabolism utilizes oxygen, provided to the tissues by the circulatory system, to convert nutrients from carbohydrates, fats, and protein into ATP. Though not as rapid as the anaerobic pathways in energy production, aerobic metabolism is efficient and certainly provides energy for much longer periods of time during moderate intensity, longer duration athletic performances. Protein is seldom used for energy production during exercise, and fats are primarily used in low intensity exercise, particularly of long duration. Thus, carbohydrates are the primary source of energy during exercise.

Carbohydrates, stored as glycogen, are present in sufficient quantities to fuel about two hours of exercise. Glycogenolysis is the process by which stored glycogen is broken down to glucose-6-phosphate that can then enter the glycolysis pathway and produce ATP. Once glycogen depletion occurs and if the fuel is not replaced, athletic performance can decrease dramatically (i.e., “hitting the wall”). If carbohydrates are not replaced, anaerobic metabolism and metabolism of fats becomes predominant again leading to lactic acid build up and diminished performance. Optimally, an athlete will “pre-load” the body with carbohydrates prior to exercise to build up glycogen stores and forestall the need for energy replacement. However, carbohydrates can and frequently need to be replaced during exercise and thus, maintenance of performance levels beyond what is possible with just the endogenous stores. Several factors related to carbohydrate delivery can be leveraged in the provision of readily digestible carbohydrates taken in appropriate quantities and at appropriate intervals to optimize their beneficial effects. It is this replacement of carbohydrates that has been the source of much research and product development to not only document the benefits of exogenous carbohydrate supplementation but also to optimize timing of ingestion, types of carbohydrates provided, and delivery form/vehicle.

### The Role of Energy Provision in the Function of Muscles and the Brain During Athletic Performance

### Muscles

Muscles use glucose, glycogen, and fatty acids for energy. When muscles are at rest, the predominant form of energy is free fatty acids (16, 17). With increasing intensity of exercise, the type of energy source changes. At low-intensity sub-maximal exercise, muscles primarily use blood glucose and free fatty acids as energy sources. As the intensity of the exercise increases, more energy is derived from glycogen and glucose, with glycogen eventually becoming the primary energy source. This use of glycogen and glucose continues until the stores are depleted. In the case of high-intensity isometric exercise, anaerobic glycolysis and the conversion of phosphocreatine to ATP are the primary energy sources (18). Given this important role of glycogen and glucose in energy provision during exercise and particularly intense exercise, it makes sense that increasing energy stores prior to exercise would be of benefit. To this end, studies have clearly demonstrated the effect of carbohydrate loading on exercise performance. For example, carbohydrate loading in trained cyclists has clearly been demonstrated to increase performance (19) as well as for other athletes (3, 20). In addition, the replenishment of energy during exercise through consumption of carbohydrate and glucose-containing energy products has also resulted in positive effects on performance (21). However, the administration of energy replenishment products during athletic performance may have unintended effects, such as gastrointestinal distress, if the volume necessary to provide replenishment is too great or if the concentration of the solution produces osmotic imbalance.

### Brain

Few human studies have been conducted to assess the role of energy supplementation/provision on the brain and cognitive function during athletic performance. However, some interesting studies in animals have provided insights regarding the potential effects of this supplementation. However, feasibility of methods for testing cognitive function and glucose uptake and effects in humans is less than in animals.

During prolonged and exhaustive exercise, in the absence of supplementation, hypoglycemia is common and brain glycogen decreases. This occurs due to an increase in brain neurotransmitters which not only induce central fatigue but also can enhance glycogenolysis in the astrocytes (22). From this finding, Matsui and colleagues (23) have hypothesized that changes in brain glycogen may play a role in the mechanism of central fatigue during prolonged exhaustive exercise. Matsui and colleagues (24) reported that in rats, during the recovery phase after exhaustive exercise, brain glycogen supercompensation occurs earlier (˜6 hr) than in either skeletal muscle or liver (˜24 hr). This finding is congruent with the “Selfish Brain Theory” put forth by Peters et al. (25), which addresses the competition for energy resources in the body and suggests that the brain will restore brain energy stores first to stave off neuronal death. A number of studies in rats have also demonstrated that increased energy demand during endurance training results in increased brain glycogen levels. This may be an important adaptation of the brain to address increased energy demands during exercise.

### Strategies for Improving Glucose Delivery and Absorption

To optimally deliver energy (e.g., glucose) during athletic performance, one must consider a number of factors including the type of carbohydrate (simple vs. complex), changes in gut blood flow and digestion during exercise, the location of release of glucose in the GI tract to optimize uptake and the rate of release of glucose.

With respect to type of carbohydrate used for energy provision and replenishment, in some embodiments of the methods of the invention, it may be optimal to utilize a mixture of simple and complex carbohydrates. It is expected that simple carbohydrates (e.g., glucose) will provide immediate energy, whereas complex carbohydrates (due to their slower processing) will provide a more sustained release of energy to the body. In certain embodiments, one may also use delivery systems that utilize only simple carbohydrates (e.g., glucose) but are able to be “tuned” to provide both an immediate release for immediate energy needs and a more sustained release to continue maintenance of energy. In embodiments employing this type of delivery system, the ease of body processing of simple carbohydrates is combined with the ability to provide both immediate and sustained energy. In addition, this type of system would also help avoid the “crash” from a bolus of glucose (energy) and resulting insulin surge that can result in a net decrease in energy and reducing the need for additional feedings.

Because one experiences both a decrease in gastric blood flow and a decrease in digestion during exercise, one should be cognizant of the volume of liquid taken in and its propensity to cause GI distress. Accordingly, in certain embodiments, it is desirable to use delivery formulations that are easily digested and provide the maximum amount of energy in the least volume and in a form that is less upsetting to the GI system.

Because absorption of glucose and subsequent release of satiety hormones occurs more readily in the distal portions of the small intestine as compared to the proximal portions closer to the stomach, in some embodiments, it is desirable to use delivery systems that can delay release slightly so that glucose is made more available in the distal segments of the small intestine in order to optimize glucose uptake. In addition, because exposure to glucose in the more distal segments can cause an increase in release of the satiety hormone GLP-1 and a decrease in the hunger hormone ghrelin, in certain embodiments, delivery to the distal small intestine provides the added benefit of reducing hunger. In yet other embodiments, since glucose (and other sugars) are transported across the gut wall by transporters, such as SGLT1 and GLUT2, delivery systems are used that maximize the exposure of these transporters to glucose by maintaining gut glucose concentrations in the region of the transporters in order to maximize energy provision.

### Use of Stimulants, Vitamins and Amino Acids During Athletic Performance

### Caffeine

Pharmacology

Though caffeine is traditionally thought of as an inhibitor of adenosine receptors (26), it has been theorized to exhibit a different mechanism of action in increasing exercise performance (27). For example, it has been proposed that caffeine may increase fat utilization and decrease glycogen utilization. This is proposed to occur through increasing circulating epinephrine levels that result in mobilization of free fatty acids and potentially intramuscular triglycerides. This increase in epinephrine may also cause the release of glucose from the liver. The central nervous system effects of caffeine are also thought to lower the neuron activation threshold, making it easier to recruit muscles for exercise. Caffeine may also increase the release of calcium from the sarcoplasmic reticulum in muscle fibers. In addition, the increases in heart rate may also serve to increase oxygen delivery to tissues. However, typically, caffeine consumption only can potentiate the use of stored energy and does not result in energy replacement. Thus, “energy” drinks or shots that contain caffeine as the principal active ingredient (and do not contain carbohydrates) do not actually provide energy. However, there is a report of concomitant consumption of caffeine with glucose-containing solutions resulting in greater oxidation of the exogenous carbohydrates as compared to the exogenous carbohydrates alone (28). In this study of cyclists who received either a glucose solution, a glucose solution plus caffeine (5 mg/kg/h), or water during exercise, subjects who received the glucose plus caffeine solution experienced 26% greater oxidation of exogenous carbohydrates as compared to subjects receiving carbohydrate alone.

### Efficacy

Muscle Effects

A large number of studies have been conducted to assess the potential benefit of caffeine consumption on athletic performance [see (29) for a comprehensive review]. A representative few will be discussed herein. Most studies have demonstrated a modest effect of caffeine consumption on athletic performance in controlled situations. However, there have been some studies published that were unable to demonstrate a positive effect. In general, it is believed that overall, caffeine consumption has a very modest positive effect on exercise performance. For example, Ivy et al., (30) observed that consumption of a caffeine (160 mg) containing drink prior to a cycling time trial reduced the time to complete the trial ˜5% (p<0.01) with no effect on perceived exertion. Cox and colleagues (31) evaluated the effect of caffeine (with various doses of caffeine given either pre- and during performance or during performance only, all at varying times and intervals) during a 2-hour steady state cycling effort followed by a time trial. All subjects were also receiving a carbohydrate containing drink both before and during the cycling study period. Co-administration of caffeine resulted in a 2-3% increase in performance depending on the dose and frequency of caffeine administration. In a study of the effects of caffeine on running performance, Bridge and Jones (32) evaluated the effect of caffeine (3 mg/kg one hour prior to running), placebo or no treatment over an 8 km run. A 1.2% (p<0.05) improvement in performance was noted in subjects receiving caffeine as compared to placebo. Conversely, a study (33) in women athletes doing repeated sprints found that administration of the same caffeine-containing energy drink as above had no beneficial effect on performance. In untrained individuals, caffeine (400 mg) (34) drink consumption did not have any positive effects on either bench press or leg extension strength or cycle ergometry. Similarly, Hunter et al. (35) conducted a trial with trained cyclists in which these athletes participated in a 100 km trial with bursts of high intensity cycling and received either placebo, a 7% carbohydrate containing energy drink, or caffeine (6 mg/kg+maintenance doses) in addition to the carbohydrate drink. No differences were noted in subjects receiving caffeine versus those that did not. What is not clear from a comparison of each of these studies is whether differences in study design may have contributed to conflicting results. As demonstrated by the above studies, the effects of caffeine are modest and variable.

### Brain Effects

In the only study published to our knowledge (36), the effects of caffeine on cognitive function was monitored both pre- and post-exercise with various doses of caffeine or placebo. In this one-hour cycle ergometry trial, trained athletes were given in a double blind fashion, energy drink containing either placebo or caffeine (150, 225 or 320 mg). Cognitive function was measured pre- and post-performance and improvements in attentional, psychomotor, and memory tests were noted. Before exercise, the energy drink with caffeine (low dose) improved long term memory. After exercise, both low and medium dose caffeine containing energy drinks improved all cognitive tests (attentional, psychomotor, and memory).

### Adverse Effects of Caffeine

Caffeine has been demonstrated to produce a number of potential adverse effects, including tachycardia, increased blood pressure, insomnia, nervousness, headaches, and arrhythmias, including during exercise performance (37). Most commonly, these adverse effects are associated with doses of 200 mg and above, but degree of exercise undertaken and age (more vulnerability to adverse effects in adolescent and teens) may impact the incidence of adverse effects of caffeine during exercise. One report (38) has suggested that doses of 6-9 mg/kg (˜420-630 mg for a 70 kg individual) can result in jitters, increased heart rate, and a diminution in exercise performance. Case reports have listed incidences of serious cardiac adverse effects in individuals consuming caffeine during exercise (e.g., see (37, 39) for a review of some cases), including arrhythmias and death. Certainly, the stimulant properties of caffeine are commonly exploited by the general population to promote wakefulness. Though caffeine consumption may have a positive effect on alertness, over-consumption can also lead to difficulty sleeping and insomnia. No conclusive studies have been reported in athletes to be able to discern whether the potential positive effects (e.g., wakefulness) are balanced by or outweighed by potential negative effects on sleep.

It is also worth a brief discussion on the effects of caffeine on hydration. Caffeine consumption can produce a diuresis, leading to speculation that this diuresis could affect hydration status. However, studies have demonstrated this effect to be modest and certainly in the case of regular caffeine consumption, a tolerance to this diuresis seems to develop [see Armstrong 2002 (40) for a review]. This effect may also be tempered by the proficiency of the kidneys in maintaining a homeostatic state. Thus, there appears to be little evidence to suggest that caffeine consumption affects hydration status. It should also be mentioned that studies (41, 42) have failed to demonstrate any significant effect of caffeine on urine production, sweat rates, or hydration status in athletes.

A recent review (29) reports that the dose of caffeine in various energy supplements may range from ˜50-500 mg per serving. This range of an order of magnitude is substantial and suggests that different formulations may produce different degrees of adverse effects and may partially explain the range of effects noted. Obviously, formulations with this amount of caffeine (˜500 mg) would provide a dose that has been demonstrated to produce adverse effects, but the consumption of multiple servings of formulations containing lower amounts of caffeine can result in similar doses.

### Vitamins, Amino Acids, Nutrients and Nutraceuticals

Vitamins and amino acids including, but not limited to vitamin B6, vitamin B12, niacin, folic acid, citicoline, phenylalanine, tyrosine, malic acid, glucuronolactone carnitine, Ginkgo biloba, Guarana, green tea, Yerba Mat, etc. have been included in various energy supplements. The hypothesis is that because these are components of cellular metabolism, supplementation (as done with carbohydrates) would increase exercise performance. However, to date, no controlled clinical trials have been conducted that demonstrate any positive effects of any of the above-listed compounds on athletic performance.

The International Society on Sports Nutrition has released a position statement on the use, efficacy, and safety of energy drinks. This group has concluded that though purported to either improve athletic performance or improve mental acuity, the primary active ingredients in these respects are carbohydrates and caffeine and that no studies have demonstrated a positive effect of any of the other agents. They also go on to caution that use of these types of drinks, especially those with caffeine or other stimulants, should only be consumed by adolescents and children with parental approval and a full understanding of the doses of ingredients and potential side effects. In addition, they suggest that indiscriminant use more than one time per day or use in people with certain pre-existing medical conditions may result in potentially harmful effects.

Studies have clearly demonstrated the positive effects of carbohydrate consumption on athletic performance. Pre-performance carbohydrate loading and supplementation with carbohydrates can have positive effects. Though various carbohydrates such as glucose, fructose, and maltodextrins are used in supplement formulations, typically, glucose provides the most direct and impactful source of carbohydrates. Though other vitamins, nutrients and other supplements, such as caffeine, have also been used to enhance athletic performance, the effects are generally very modest and variable. Furthermore, one should be cautious with caffeine consumption in larger doses as significant adverse effects have been reported. Thus, in certain embodiments, the use of carbohydrates as a supplement in the methods of the instant invention is the most beneficial form of supplementation during athletic performance with a delivery system providing carbohydrates/glucose in sufficient quantities and at rates that would optimize uptake and utilization by the body in formulations that do not produce unwanted effects, such as GI distress.

As described herein, in certain embodiments, the instant invention provides methods and related compositions for improving cognitive function. For example, in certain embodiments, the methods of the instant invention provide energy supplementation and/or provision to the brain of an individual such that cognitive function is improved in the individual. In other embodiments, the instant invention provides methods and related compositions for treating a central nervous system (CNS) disease or condition. For example, by providing energy supplementation and/or provision to the brain, and in particular, to one or more nerve cells of the brain, CNS diseases and conditions such as ischemia, neurodegenerative disorders, mental health disorders, pain disorders, addiction disorders, brain or spinal cord injuries, and/or brain or spinal cord tumors can be treated.

In yet other embodiments, the instant invention provides methods and related compositions for treating a metabolic disorder. For example, in certain embodiments, the instant invention provides methods and related compositions for delivering glucose to the small intestine such that the glucose is delivered to glucose transporters, such as SGLT1 and GLUT2, over an extended period of time, thereby controlling for glucose absorption and maintenance of blood glucose levels. Metabolic disorders that can be treated according to the methods described herein include obesity, metabolic syndrome, and hypoglycemia. In the case of hypoglycemia, for example, in certain embodiments, the methods of the invention can result in both an immediate rise in blood glucose and also a sustained increase in blood glucose. This is beneficial to assist care-givers/first-responders in providing a means to raise blood glucose and keep it elevated near more normal levels prior to arrival at emergency departments and thus, reduce the chance of brain damage that occurs with prolonged hypoglycemia.

In further embodiments, the inventive methods described herein relate to methods and related compositions for hormone modulation, such as satiety and/or hunger hormone modulation. For example, in certain embodiments, the instant invention provides methods and related compositions for delivering glucose to the small intestine such that blood glucose levels are increased, resulting in increased levels of one or more satiety hormones, such as colecystokinin (CCK) and glucagon-like peptide 1 (GLP-1), and/or decreased levels of one or more hunger hormones, such as ghrelin. Other examples of satiety hormones that may be modulated include peptide YY (PYY), pancreatic polypeptide (PP), insulin, and incretins, including in addition to GLP-1, oxyntomodulin and glucose-dependent insulinotropic polypeptide.

In yet other embodiments, the instant invention provides methods and related compositions for treating a metabolic disorder, wherein the disorder is insulin resistance, hyperglycemia, impaired glucose tolerance, and/or diabetes, such as type 1 diabetes, type 2 diabetes, gestational diabetes, and MODY (maturity onset diabetes of the young).

Treatment for different diseases and conditions as described herein is generally accomplished by administration of an active agent, such as an energy supplement in the form of, e.g., a carbohydrate such as glucose, to an individual in need thereof via a delivery system that delivers the active agent, such as an energy supplement, to the gastrointestinal tract of the individual, and in particular, to the distal segments of the intestinal tract.

The intraluminal pH is rapidly changed from highly acidic, pH 2, in the stomach to about pH 6 in the duodenum. The pH gradually increases in the small intestine from pH 6 to about pH 7.4 in the terminal ileum. The pH drops to 5.7 in the caecum, but again gradually increases, reaching pH 6.7 in the rectum. See, e.g., Evans, D F, et al. Gut (1988) 29:1035-1041.

In some embodiments, delivery of an active agent, such as a molecule from the Biopharmaceutics Classification System (BCS) categories of BCS I, BCS II, or BCS III (see, e.g., Folkers, G, et al. (2003) Drug Bioavailability: Estimation of Solubility, Permeability, Absorption and Bioavailability (Methods and Principles in Medicinal Chemistry). Weinheim: Wiley-VCH; Amidon, G L, et al. (1995) Pharm. Res. 12 (3):413-420, incorporated by reference herein; and the U.S. Food and Drug Administration website regarding BCS guidance (e.g., www(dot)fda(dot)gov/AboutFDA/CentersOffices/OfficeofMedicalProductsandTobacco/CDER/u cm128219(dot)htm)) is delivered to specific portions of the intestine and in proximal contact with specific transporters to improve delivery of the drug to the systemic circulation. In these cases, the improved bioavailability of the active agent (e.g., BCS Class I, II, or III) results in an improved therapeutic effect.

In certain embodiments, delivery of an active agent, such as a molecule from the Biopharmaceutical Class System (BCS) categories of BCS I, BCS II or BCS III is delivered to specific portions of the intestine to produce local effects and treat intestinal disorders. These disorders may include diarrhea, constipation, intestinal infection, Crohn's disease, and inflammatory bowel disease. In addition, in certain embodiments, the active agent may be delivered either orally (e.g., a beverage or chew formulation) or rectally (e.g., an enema formulation) according to the methods of the invention.

Accordingly, the methods of the instant application are applicable to a wide range of active agents. Non-limiting examples of BCS active agents for use in the inventive methods described herein include metformin, levodopa, phenylalanine, pravastatin, cimetidine, methotrexate, theophylline, and zidovudine.

In particular embodiments, the active agent is combined with a sugar through glycosylation. This glycosylated active agent is then delivered to sections of the intestine containing SGLT and/or GLUT transporters, wherein the glycosylated bioactive is actively transported into the circulation. Once inside the systemic circulation, the sugar may be cleaved to release the nonglycosylated active agent or if the glycosylated form of the agent is active, then it may remain intact to produce the desired effect.

In particular embodiments, delivery of an active agent, such as a carbohydrate, to a distal portion of the small intestine, enables the controlled delivery of the active agent to target cells and receptors of the intestinal epithelium. In certain embodiments, where the active agent is a carbohydrate such as glucose, controlled delivery of the glucose to distal regions of the small intestine provides prolonged exposure of glucose transporters, such as SGLT1 and GLUT2, to the glucose, thereby resulting in increased absorption of the glucose from the small intestine into the circulatory system. In certain embodiments, this provides for treatment of metabolic disorders where an improvement in glucose regulation is needed.

At the same time, calibrating the delivery of glucose to the small intestine, in particular, to distal portions of the small intestine, provides for the modulation of hormones such as satiety and/or hunger hormones. For example, in one embodiment, an individual can be treated for obesity by increasing glucose absorption in distal segments of the small intestine through a method of the instant invention, resulting in an increase in the generation of satiety hormone levels, thereby providing feelings of fullness and satiation in the individual, resulting in reduced food intake. Similarly, by increasing glucose absorption in the distal segments of the small intestine through a method of the instant invention, levels in hunger hormones, such as ghrelin, can be reduced, resulting in reduced food intake in an individual.

In embodiments where the active agent to treat a CNS disease or condition or to improve cognitive function is a carbohydrate such as glucose, in certain embodiments, delivery of the glucose to distal portions of the small intestine and subsequent glucose absorption results in and improves the uptake of glucose and allows more glucose to be made available for uptake into the brain. In particular embodiments, delivery of glucose to improve cognitive function results in an increase in brain glycogen stores. In certain embodiments, improvements in cognitive function include improvements in attention, psychomotor, and/or memory abilities.

As used herein, the terms “drug,” “agent,” and “compound” encompass any composition of matter or mixture which provides some pharmacologic effect that can be demonstrated in vivo or in vitro. This includes small molecules, nucleic acids, proteins, antibodies, vaccines, vitamins, and other beneficial agents and bioactive substances. As used herein, the terms further include any physiologically or pharmacologically active substance that produces a localized or systemic effect in a subject (e.g., a mammal, such as a human).

Therapeutic agents suitable for use in the methods and delivery systems of the instant invention include but are not limited to chemotherapeutic agents, steroids, retinoids, antimicrobial compounds, antioxidants, anti-inflammatory compounds, vitamin D analogs, salicylic acid, NMDA receptor antagonists, endothelin antagonists, immunomodulating agents, angiogenesis inhibiting/blocking agents, compounds inhibiting FGF, VEGF, EGF or their respective receptors, tyrosine kinase inhibitors, protein kinase C inhibitors, and combinations thereof. A therapeutic agent includes pharmaceutically acceptable salts thereof, prodrugs, and pharmaceutical derivatives thereof.

The term “antimicrobial compound” relates to any compound altering the growth of bacteria, fungi, or viruses whereby the growth is prevented, modified, reduced, stabilized, inhibited, or stopped. Antimicrobial compounds can be microbicides or microbiostatic agents and include but are not limited to antibiotics, semi-synthetic antibiotics, synthetic antibiotics, antifungal compounds, antiviral compounds and the like.

Active agents for use in the methods of the instant invention also include carbohydrates, proteins, amino acids, vitamins, co-enzymes, phospholipids, minerals, and electrolytes. Examples of vitamins and co-enzymes that may be delivered using the methods of this invention include but are not limited to water or fat soluble vitamins such as thiamin, riboflavin, nicotinic acid, pyridoxine, pantothenic acid, biotin, flavin, choline, inositol and paraminobenzoic acid, carnitine, vitamin C, vitamin D and its analogs (such as ergocalciferol, calcitriol, doxercalciferol, and paricalcitol), vitamin A and the carotenoids, retinoic acid, vitamin E and vitamin K.

In certain embodiments, the methods of the invention provide for the delivery of carbohydrates that are taken up by different receptors, e.g., SGLT and GLUT receptors. Suitable carbohydrates include, but are not limited to, mono-, di- and polysaccharides such as glucose, sucrose, maltose as well as more complex edible carbohydrates such as maltodextrins. Examples of suitable carbohydrates also include dextrose, fructose, galactose, lactose, polydextrose, dextrins, corn syrup solids, starch, and combinations thereof. Important digestible carbohydrates include: the monosaccharides—glucose, fructose and galactose; the dissacharides—sucrose, maltose and lactose; and the polysaccharide, starch. Starch is broken down in to dextrins by salivary amylase (in the mouth) and pancreatic amylase (in the small intestine). Dextrin is acted upon by the brush border enzymes in the small intestine, which also convert the double sugars into simple sugars. The monosaccharides are finally transported across the intestinal epithelium into the bloodstream. In certain embodiments, the treatment methods of the instant invention provide for the controlled release of digestible carbohydrates, especially the simple sugars, glucose and fructose, for sustained uptake into the blood.

According to one embodiment, a composition for use in the methods of the invention includes a blend of glucose and fructose. In certain embodiments, the weight ratio of glucose to fructose ranges from about 1:1 to about 100:1, about 5:1 to about 95:1, about 10:1 to about 90:1, about 15:1 to about 85:1, about 20:1 to about 80:1, about 25:1 to about 75:1, about 30:1 to about 70:1, about 35:1 to about 65:1, about 40:1 to about 60:1, about 45:1 to about 55:1 or about 50:1. In certain embodiments, the composition includes from about 0.1 to about 99.9 wt. %, about 1 to about 99 wt. %, about 5 to about 95 wt. %, about 10 to about 90 wt. %, about 15 to about 85 wt. %, about 20 to about 80 wt. %, about 25 to about 75 wt. %, about 30 to about 70 wt. %, of carbohydrates, about 35 to about 65 wt. %, about 40 to about 60 wt. %, about 45 to about 55 wt. %, or about 50 wt. %, calculated on a 100% dry matter basis of the composition.

The rate and extent of exogenous carbohydrate absorption may be limited not only by the amount of carbohydrate available but also by the maximum intestinal transport capacity for glucose and fructose. As discussed above, intestinal transport of glucose is mediated by a sodium dependent glucose transporter (SGLT1), located in the brush-border membrane. SGLT1 transporters may become saturated at a glucose ingestion rate of about 1 g/min. Fructose on the other hand is absorbed from the intestine by GLUT-5, a sodium-independent facilitative fructose transporter. Generally, ingestion of a mixture of carbohydrates that have different transport mechanisms for absorption into the bloodstream, simultaneously increases carbohydrate and water absorption.

In other embodiments, the methods of the instant invention provide for the delivery of amino acids. The amino acids may be in the foam of free amino acids or peptides, and in certain embodiments, are present in an amount in the range of from about 0.1 to about 99.9 wt. %, about 1 to about 99 wt. %, about 5 to about 95 wt. %, about 10 to about 90 wt. %, about 15 to about 85 wt. %, about 20 to about 80 wt. %, about 25 to about 75 wt. %, about 30 to about 70 wt. %, of carbohydrates, about 35 to about 65 wt. %, about 40 to about 60 wt. %, about 45 to about 55 wt. %, or about 50 wt. % calculated on a 100% dry matter basis of the composition.

The peptide material can be derived from proteins of animal or plant origin and examples of such proteins are milk proteins, meat proteins, soy proteins, wheat proteins, pea proteins, rice proteins and maize proteins. In some embodiments, the protein raw material is wheat gluten protein or a subfraction thereof such as gliadin. In the present context, the term “peptide material” is understood to indicate a protein hydrolysate and may contain all types of peptides that may vary in length as well as a certain amount of free amino acids resulting from the hydrolysis. The protein raw material is hydrolyzed by one or more hydrolytic enzymes. The hydrolytic enzyme can be of animal, plant, yeast, bacterial or fungal origin. In certain embodiments, enzyme preparations are used which have a low exo-peptidase activity to minimize the liberation of free amino acids and to improve taste profiles of the protein hydrolysates. In particular embodiments, hydrolyzed protein material employed in the methods of the present invention has an average peptide chain length in the range of 1-40 amino acid residues and in certain embodiments, in the range of 1-20 amino acid residues. The average peptide chain can be determined using the method as described in WO 96/26266. Further, the peptide material can be present in an amount of about 0.1-90 wt. %, calculated on dry matter basis of the composition.

Other optional components of the compositions delivered according to the methods of the instant invention are vitamins, minerals, electrolytes, flavors, antioxidants, components having co-enzyme and antioxidant properties, lipids including emulsifiers, and proteins for meeting specific nutritional and/or physiological needs.

An active agent, such as a carbohydrate, e.g., dextrose, fructose, and the like and combinations thereof, may be present in a composition for use in the methods of the invention in any desirable amount, including, for example, about 1-20 wt. % of the composition, e.g., 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, or 20 wt. % of the composition. Also 20-25 wt. %, 25-30 wt. %, 30-35 wt. %, 35-40 wt. %, 40-45 wt. %, 45-50 wt. %, and greater than 50 wt. %.

In another embodiment, different types of carbohydrates, e.g., those that are taken up by, for example, SGLT transporters versus GLUT transporters, are added in differing ratios at differing release rates to achieve the results as described infra.

The treatment methods of the instant invention provide for the delivery of one or more active agents by administration of a composition comprising the one or more active agents. The compositions employed in the treatment methods of the instant invention typically comprise particles that are microparticles (e.g., 1-1000 micrometers in diameter) and/or nanoparticles (e.g., 1-1000 nanometers in diameter) and that contain the one or more active agents, e.g., encapsulated or integrated therein. In certain embodiments, the compositions employed in the treatment methods of the instant invention comprise particles below 100 micrometers in size. Delivery vehicle systems especially suited to the methods of the instant invention are described in U.S. Pat. No. 8,563,066 and U.S. Patent Application Publication No. 2012/0015039, both of which are incorporated herein by reference.

In certain embodiments, the microparticles contain multiple layers designed to obtain release kinetics comprising the sequential release of two or more active agents. In further embodiments, different layers of the particles contain different active agents, which are released in such a manner that the peak concentrations of these agents are separated (or resolved) in time.

In certain embodiments, the microparticles are delivered as a gel that is suitable for oral, trans-mucosal (including buccal, intranasal, rectal), topical, transdermal, and/or intradermal suspensions for intra-cavity administration.

The term “sustained release” (i.e., extended release and/or controlled release) are used herein to refer to an active agent, for example carbohydrates, etc., delivery system or composition that is introduced into the body of a subject (e.g., a mammal, such as a human) and that continuously releases a stream of one or more active agents over a predetermined time period and at a level sufficient to achieve a desired effect throughout the predetermined time period. Reference to a continuous release stream is intended to encompass release that occurs as the result of diffusion-limited release of the component from the matrix, or biodegradation in vivo of the composition, or a matrix or component thereof, or as the result of metabolic transformation or dissolution of the added active agent(s) or other desired agent(s). Delayed release may be achieved by entrapping the active agents within particulate carriers with mucoadhesive surface characteristics. Adhesion of the active agent-loaded particles to intestinal mucosa will increase retention time of the particles inside the intestinal lumen, thereby providing continuous release and transport of active agents across the epithelium into blood, beyond the normal retention time of non-adhesive composition inside the gastrointestinal tract.

In one embodiment, the active agent composition is in the form of a solution, suspension, gel capsule, gel matrix (e.g., a chew), powder, snack (e.g., a bar), granola fowl, or tablet. The “delivery” of active agents comprises, for example, suspending the active agents individually or in combinations in sustained release particulate particles (e.g., microparticles), compounds which bind to the active agents with different affinities and the like. According to one embodiment, the requisite volume for consumption by the individual is about 500 mL when in liquid form; however, formulations increasing and or decreasing the concentrations and amounts are contemplated. For example, in certain embodiments, the volume for consumption is less than or equal to 150 mL when in liquid form.

In another embodiment, the active agents are present in nano suspensions/colloidal particles. The nanoparticles or colloidal particles (CP) can form a stable colloidal suspension in water and in a physiological medium. The CP associate with the active agents, e.g., carbohydrates, in aqueous media by a spontaneous mechanism, and the CP release the active agents in a physiological medium and, more precisely, in vivo. The release kinetics depend on the nature of the polymer that is the CP precursor. A protein, whose pharmaceutical or nutritional value depends on the tertiary structure of the molecule may also be delivered by this method, using biocompatible polymer hosts that will not denature the protein.

Thus, by varying the specific structure of the polymers, it is possible to control the association and release phenomena from the kinetic and quantitative points of view.

Another embodiment of the invention concerns the preparation of: selected particles; and other selected particles which are structured, submicron and capable of being used especially for carrying one or more active agents (e.g., bioactives), these particles being individualized (discrete) supramolecular arrangements that are: based on linear amphiphilic polyamino acids having peptide linkages and comprising at least two different types of hydrophilic repeating amino acids, and hydrophobic repeating amino acids, the amino acids of each type being identical to or different from one another; capable of associating at least one active agent in colloidal suspension, in the undissolved state, and releasing it, especially in vivo in a prolonged and/or delayed manner; and stable in the aqueous phase at a pH of between 4 and 13, in the absence of surfactant(s).

In certain embodiments, the particles are submicron structured particles capable of being used especially for carrying one or more active agents, these particles being discrete supramolecular arrangements; capable of associating at least one active agent in colloidal suspension, in the undissolved state, and releasing it, especially in vivo, in a prolonged and/or delayed manner; and stable in the aqueous phase at a pH of between 4 and 13, in the absence of surfactant(s).

In another embodiment, the composition can be formulated to encapsulate the active agent compositions in microspheres or microparticles so that it may be admixed or formulated into any form, such as a powder, gel, a beverage, gum, nutritional food product, pill and the like. In certain embodiments, compositions of the invention are formulated to comprise one or more active agents in a chew, for example, where the one or more active agents are formulated into micro- and/or nanogel particles that are admixed or formulated into a liquid center located within a solid or semisolid gel matrix.

Further to the above, suitable foams for delivery of active agents according to the instant invention include oral, trans-mucosal (including buccal, intranasal, rectal), topical, transdermal, and intradermal, suspensions for intra-cavity administration, as well as suspensions for bathing organs during transplant activities. In certain embodiments where trans-mucosal delivery is desired, the pH- and temperature-responsive delivery systems described herein will allow tuning of drug delivery based on predicted temperature and pH at the specific biological environment site targeted. In embodiments where topical delivery is desired, the hydrogels as described herein (e.g., microgels) could be incorporated into a carrier gel that may be applied directly to the dermis after which drug delivery would be controlled by appropriate particle release kinetics and dermal flux rate. With respect to transdermal application, in certain embodiments, transdermal application of an active agent would function in a similar manner. In further embodiments, a drug delivery formulation containing microgels of the instant application could be used in the “reservoir” of a transdermal patch, thereby providing additional control over burst release and ongoing bioactive delivery through the skin.

Regarding intradermal application, in certain embodiments, a formulation incorporating microgels of the instant application is delivered directly to subcutaneous tissue, incorporated as payload into an implantable instrument such as a bioresorbable disk, tube, and the like from which the microgels would react to the pH and temperature of the immediate environment. Such methods of use in the area of intra-cavity administration provides an advantage of delivering bioactive substances directly to an infected region or specific organ in vivo and provides large surface area coverage and controlled and extended release of bioactive substances as may be required in certain surgical and traumatic open-cavity wound-care situations. In certain embodiments, use of suspensions of microgels, loaded with the appropriate bioactive and bioprotective substances, in bathing transplant organs is expected to aide in organ viability and improved transplant success rates.

A “microsphere” or “microparticle”, as defined herein, includes a particle of a biocompatible solid-phase material having a diameter of about one millimeter to about one micrometer (micron), or less, wherein the particle may contain a biologically active agent and, wherein the solid-phase material sustains the in vivo release of the active agents from the microsphere. A microsphere can have a spherical, non-spherical, or irregular shape. The typical microsphere shape is generally spherical.

A “nanosphere” or “nanoparticle”, as defined herein, includes a particle of a biocompatible solid-phase material having a diameter of about one micrometer to about one nanometer, or less, wherein the particle may contain a biologically active agent and, wherein the solid-phase material sustains the in vivo release of the active agents from the nanosphere. A nanosphere can have a spherical, non-spherical, or irregular shape. The typical nanosphere shape is generally spherical.

A “biocompatible” material, as defined herein; means that the material, and any degradation products of the material, is non-toxic to the recipient in the concentration(s) administered to a subject, and also presents no significant deleterious or untoward effects on the recipient's body.

In one embodiment, the microspheres contain a mixture of active agents, and the microsphere is composed of a biodegradable material that is released over a certain period of time. For example, in order to provide an initial burst of active agents to provide an immediate reservoir of, e.g., energy or nutrients to the individual, the active agents are formulated as such and can contain a variety of carbohydrates, amino acids, electrolytes, vitamins, etc. in differing ratios. The second group can contain a differing ratio of active agent(s) (e.g., carbohydrates:amino acids:vitamins etc.), or strictly different or similar active agents that are released over a longer period of time to maintain a sustainable release of the active agents. The formulation of the active agents in the microspheres and the timing of release can be varied depending on the types of activity, the individual, age, weight and nutritional needs. For example, a marathon runner (sustained nutrition over long period) would have different nutritional needs to a sprinter (burst of nutrition).

In another embodiment, compositions comprise compounds that dissolve over a period of time in vivo sequentially in acid, neutral and weak alkaline regions of the gastrointestinal tract. These compounds include for example, an acidic polymeric dispersion coating as the first coating to prolong active agent release. In this embodiment, the microparticle comprises as a core a material comprising calcium carbonate, sugar, dextrose and nonpareil seeds. The first coating is a material which retards rapid passage of water. The first coating is preferably an aqueous dispersion of poly(methacrylic acid-co-ethyl acrylate) (commercially available under the designation Eudragit L30D-55). The second coat is a latex acrylic polymer. The second coating is preferably poly(ethyl acrylate-co-methyl methacrylate-co-2-trimethylammonioethyl methacrylate chloride) (commercially available under the designation Eudragit RS-30D). The thickness of the second coating is established to achieve the desired time-release rate for the drug.

The time-release products are typically substantially spherical in configuration. The diameter of the time release drug products typically ranges between 1 and 650 microns, between 20 and 500 microns or between 40 and 350 microns and in some embodiments, is preferably between about 50 and 250 microns when the products are in a liquid suspension form. In certain embodiments, the time release active agent composition containing products of the present invention, because of their size, can be suspended in an aqueous medium, thereby providing a liquid suspension.

In certain embodiments, the active agent compositions are formulated as a time release formulation comprising: a core which can be optional; active agent bound to the core; a first coating having limited permeability to water; and a second coating, which is more permeable to water than the first coating, wherein the first and second coatings together comprise the time release components of the active agent compositions.

The core will generally have a diameter of about 20 to 60, about 23 to 55, about 26 to 50, or about 30 to 45 microns. The core is generally comprised of an inert ingredient, preferably a material selected from the group consisting of calcium carbonate, sugar, dextrose and nonpareil seeds.

The first coating, which has a limited permeability to water and which retards rapid passage of acid and water. This first coating will typically have a diameter of between about 1.00 and 5.00, about 1.50 and 4.50, about 2.00 and 4.00, or about 2.50 and 3.50 microns. In some embodiments, the first coating is an acidic polymeric dispersion coating which prolongs drug release, such as an aqueous dispersion of poly(methacrylic acid-co-ethyl acrylate). Such a polymer is commercially available under the name EUDRAGIT L30D-55. The core and first coating together typically have a diameter of between about 60 and 80, about 62 and 75, or about 65 and 70 microns.

It is appreciated that the first and second coatings together comprise the time release components of a product of the present invention. The first and second coatings together effect time release of an orally administrable drug within an individual over a maximum period of about 12 hours. It is appreciated by those ordinarily skilled in the art that the thickness of the second coating can be altered to achieve the desired time release rate for the active agent. That is, the thickness of the second coating can be increased to achieve a longer period of time release in the body. The coatings work due to differential porosity. The inner coating comprised of, for example, poly(methacrylic acid-co-ethyl acrylate) is sensitive to pH. Active agent transport across the inner coating can be determined by the porosity and water content of the coating, both of which can be determined by the different pH values within regions of the gastrointestinal tract. In an acidic environment (e.g., in the stomach), the inner coating becomes relatively hydrophobic and shrinks, leading to decreased pore size and active agent permeability. In contrast, the pH inside the intestinal lumen is higher. The inner coating becomes relatively hydrophilic due to ionization, and allows faster release of active agents from the particle cores. In certain embodiments, the outer coating is not pH-responsive, but can be used to control active agent permeability by controlling the pore size. The present invention provides in these embodiments where the first and second coating porosity are such that water entering the time release component will pass through the second coating more rapidly than through the first coating and the drug and water exiting the time-release component will pass through the first coating more slowly than through the second coating. In one embodiment, passage through each coating is by mechanical means with the passage through the first coating being augmented by ionic interaction.

In another embodiment, one or more of active agents are bound or encapsulated by a particle, which is stable in an aqueous environment and are released over an extended period of time once the active agents have been consumed.

The composition according to the invention may have the form of a powder, gum, a beverage or any other food product. A beverage according to the invention can be prepared by dissolving the above-defined ingredients in an appropriate amount of water. In some embodiment, an isotonic drink is prepared. For drinks intended to be used during and after exercise, it is recommended to have a concentration of the composition according to the invention in the range of about 0.10-60 wt. % calculated on the total weight of the drink.

In one embodiment, the formulation has a viscosity and “mouth-feel” similar to liquids. The viscosity determined at room temperature using a cup and cylinder rheometer (e.g., Discovery Hybrid rheometer, TA Instruments) can be in the range of 2000 cP to 1 cP, over a shear rate of 10 s⁻¹ to 1000 s⁻¹ at room temperature. The viscosity can vary between about 1500 cP and about 1 cP over a temperature range of 25° C. to 60° C. Room temperature viscosity of water is about 1 cP, while that of olive oil is about 80 cP, castor oil about 1000 cP, and corn syrup about 1400 cP. The viscosity of fat-free milk is about 30 cP [Vesa, T. H.; Marteau, P. R.; Briet, F. B. et al. Am. J. Clin. Nutr. 1997, 66, 123-126].

In some embodiments, the active agent compositions are admixed with a biodegradable binder or encapsulated within a biodegradable microsphere which allows for sustained release of desired active agents (e.g., carbohydrates and other nutrients). “Biodegradable”, as defined herein, means the polymer will degrade or erode in vivo to form smaller chemical species. Degradation can result, for example, by enzymatic, chemical and/or physical processes. Suitable biocompatible, biodegradable polymers include, for example, polysaccharides, poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and polyorthoester, biodegradable polyurethanes, hydrogels, blends and copolymers thereof.

Biocompatible, non-biodegradable polymers suitable for the methods and compositions of the present invention include non-biodegradable polymers selected from the group consisting of polyacrylates, polymethacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, hydrogels, blends and copolymers thereof.

In another embodiment, hydrogels are used in the sustained release of the one or more active agents. Physical polymeric hydrogels have been widely explored for biomaterials applications. Examples include hydrogels formed by complexation of enantiomeric polymer or polypeptide segments and hydrogels with temperature- or pH-sensitive properties. They attract special attention for sustained drug delivery because of the mild and aqueous conditions involved in trapping delicate bioactive agents such as proteins. For example, in situ formed hydrogels, formed from thermosensitive block copolymers, have also been proposed as sustained release matrices for drugs. They have the advantage that there is no chemical reaction involved in the gel formation. These copolymer hydrogels are usually designed for macromolecular drugs such as proteins and hormones. In certain embodiments, the polymer is in an aqueous solution, which forms a hydrogel. For example, suitable aqueous polymer solutions contain about 1% to about 80%, about 2% to about 75%, about 3% to about 70%, about 4% to about 65%, about 3% to about 70%, about 4% to about 65%, about 5% to about 60%, about 6% to about 55%, about 7% to about 50%, about 8% to about 45%, about 9% to about 42% polymer, about 10% to about 40% polymer. Suitable hydrogels can also contain about 1% to about 20%, about 2% to about 19%, about 3% to about 18%, about 4% to about 17% cyclodextrin (w/w) (based on the weight of total solution), about 5% to 15% cyclodextrin, to solubilize active agents that have limited water solubility. The hydrogel is typically formed using an aqueous carrier fluid. For example, typical aqueous solutions contain about 1% to about 80%, about 2% to about 75%, about 3% to about 70%, about 4% to about 65%, about 3% to about 70%, about 4% to about 65%, about 5% to about 60%, about 6 % to about 55%, about 7 % to about 50%, about 8% to about 45%, about 9% to about 42% polymer, about 10% to about 40% polymer.

The hydrogel composition may also contain a secondary polymer, which may complex with the active agent, conjugate the active agent, or both. The secondary polymer may suitably be a polyester, polyurethane, polyamide, polyether, polysaccharide, poly(amino acid), polypeptide, or a protein. In some embodiments, the secondary polymer is a di- or mono-functional polymer or polyionic polymer with poly(ethylene glycol) segments. In the case where active agents conjugate or complex to the hydrogels, then the hydrogel formulations act not only as a matrix but also a carrier of the active agents. This means that the active agents, e.g., a variety of carbohydrates, are not only physically entrapped in the hydrogel, but also are complexed or conjugated to the molecules that form the hydrogel. A secondary polymer may also be used to alter the properties, such as porosity and viscosity, of the hydrogel matrix.

The properties of the hydrogels are tunable by using block copolymers with different block molecular weights and hydrophobicity, e.g., by adjusting the cyclodextrin content, and through the use of secondary polymers. For example, the hydrogel may be adjusted to be a more flexible hydrogel or a more stiff hydrogel, as characterized by rheological measurements of storage modulus values. The hydrogel structure can be tailored to have variable viscosity (e.g., characterized by rheological measurements of loss modulus values) and longer or shorter drug release rates.

The duration of extended release is dependent on the molecular weights of the block polymers, particularly the molecular weight of the hydrophobic poly(hydroxyalkanoate) section (e.g., PHB). The release rate may be altered in accordance with the invention to achieve a desired duration of response by selecting: a particular poly(hydroxyalkanoate); the stereo-isomeric state of the selected poly(hydroxyalkanoate); the molecular weight of the selected poly(hydroxyalkanoate); and the relative quantity of cyclodextrin used in the hydrogel, to achieve a desired duration and rate of sustained release. The molecular weight and selection of the hydrophilic poly(alkylene oxide) also impacts the sustained release kinetics, but to a lesser extent than the hydrophobic poly(hydroxyalkanoate) component. Secondary polymers may also be utilized to change the release kinetics. Hydrogels can provide sustained release over a period of one or more days by adjustment of the molecular weights of the block polymers and the copolymer, as well as, e.g., the cyclodextrin content within the hydrogel of certain embodiments of the present invention and the potential use of secondary polymers.

Microencapsulation of components of the active agent in biodegradable polymers such as polylactide-polyglycolide is also contemplated. Depending on the ratio of component to polymer, and the nature of the particular polymer employed, the rate of component release may be sustained. Examples of other biodegradable polymers include poly(orthoester)s and poly(anhydride)s. The formulations can also be prepared by entrapping the component in liposomes or microemulsions that are compatible with body tissue.

Further, the terminal functionalities of a polymer can be modified. For example, polyesters may be blocked, unblocked or a blend of blocked and unblocked polymers. A blocked polyester is as classically defined in the art, specifically having blocked carboxyl end groups. Generally, the blocking group is derived from the initiator of the polymerization and is typically an alkyl group. An unblocked polyester is as classically defined in the art, specifically having free carboxyl end groups.

In an advantageous embodiment, blends of polysaccharides are utilized to synthesize aqueous dispersions of microparticles or nanonparticles. Advantageously, the polysaccharides are hydrophobically modified polysaccharides wherein the polysaccharides form interpenetrating polymer networks. In an especially advantageous embodiment, the polysaccharides contain carboxylic acid groups.

Without being bound by theory, it is expected that the carboxy containing hydrogel particles are in a collapsed state in the acidic environment of the stomach. Hence, the one or more encapsulated active agents are retained within the particles in the stomach. The hydrogel particles will achieve an expanded state when they reach the small intestine (pH 5-7), and will release the encapsulated active agent(s) at a rate faster than that in the stomach. A feature of the proposed polysaccharide hydrogels is their pH responsiveness. Ideally, the hydrogels should not swell in the acidic environment of the stomach, but should swell upon entry into the small intestine and release the encapsulated active agent(s) at a controlled rate. In certain embodiments, the active agents (e.g., carbohydrates) of the present invention are controlled release particles dispersed in an aqueous medium. but may also be stored in a solid particulate form.

In a particularly advantageous embodiment, the hydrogels comprise hydrophobized polysaccharides. Polysaccharides may be functionalized with hydrophobes such as cholesterol. For example, polysaccharides such as, but not limited to, pullulan, dextran, and mannan may be partly substituted by various hydrophobic groups such as, but not limited to, long alkyl chains and cholesterol.

The nanoparticles or microparticles of the present invention may comprise modified starch molecules with grafted fatty acid moieties. The fatty acid may be grafted on to starch using potassium persulfate, for example, as a catalyst. In another embodiment, the invention also encompasses surface-modification of nanoscale starch particles using, for example, stearic acid chloride (a hydrophobe), poly(ethylene glycol), or methyl ether (a hydrophilic molecule). In another embodiment, the modified starch may be an acryloyl-modified starch or an acryloyl-modified hydroxyethyl starch.

In an advantageous embodiment of the invention, the polysaccharide is first derivatized to introduce aldehydic or carboxylic groups on the side chain These groups are then crosslinked to produce more stable three-dimensional networks.

In an advantageous embodiment, the particles are crosslinked to form hydrogels. Crosslinking may be performed using free radical initiators such as persulfate salts, or redox systems involving ascorbic acid, or a naturally occurring crosslinker such as genipin. Ionic crosslinking may also be performed. Anionic polysaccharides such as gellan can be used for ionic crosslinking, instead of chemicals such as borax which may not be desirable in a food formulation.

The present invention further relates to the preparation of hydrogels. In an advantageous embodiment, a blend of hydrophobically modified polysaccharide such as, but not limited to, hydroxypropyl cellulose, methyl cellulose, ethyl cellulose, hydroxypropyl methyl cellulose, hydroxyethyl methyl cellulose, ethyl hydroxyethyl cellulose, methyl ethyl hydroxyethyl cellulose, hydroxyethyl cellulose, and/or cellulose acetate and a carboxy containing polysaccharide such as, but not limited to, alginate or carboxymethyl cellulose may be used to prepare the hydrogel particles of the present invention. Examples of suitable alginates include sodium alginate polymers (e.g., sodium alginate NF, F-200, SAHMUP and sodium alginate NF, SAHMUP), which may be present in a composition according to the invention in an amount of e.g., about 0.01 wt. % to about 1.0 wt. % of the composition.

The hydrophobically modified polysaccharide results in spontaneous particle formation due to phase separation in water, while the polysaccharide containing carboxylic acid groups imparts a pH-responsive behavior and will also increase intestinal transit time. In one embodiment, nanoparticle suspensions may be synthesized by self-assembly of chitosan and carboxymethyl cellulose hydrolysates. The polymers are hydrolyzed with the enzymes chitosanase and cellulase, respectively. Electrostatic interactions between the carboxylate groups of carboxymethyl cellulose with the amino groups of chitosan result in spontaneous formation of nanoparticles by mixing solutions of the two polymers. Particle size depends on the mixing ratio of the solutions, and also by the molecular weight of the polymers. In some embodiments, it may be necessary to hydrolyze the polymers and lower the molecular weight before mixing in order to prevent the formation of macroscopic gel.

In another embodiment, hydrogels may be prepared from mixtures of acidic polysaccharides such as, but not limited to, alginates, and basic polysaccharides such as, but not limited to, oligosaccharide derivatives of chitosan; a basic polysaccharide such as, but not limited to, chitosan and anionic polysaccharide such as, but not limited to, hyaluronic acid; alginate and oxidized alginate blended with chitosan; grafted agar and sodium alginate blend with acrylamide; gellan co-crosslinked with scleroglucan; photocrosslinked modified dextran; starch reacted with glycidyl methacrylate; or polymerizable saccharide monomers, such as sucrose, created by reaction of the sugar with epoxy acrylate, or methacryloyl chloride and acetyl chloride.

Crosslinking of polysaccharides containing hydroxyl groups, e.g., starch, hydroxyalkyl starch, hydroxyalkyl cellulose, etc., can be achieved using a variety of reagents including bis-epoxides, divinyl sulfone, N,N′-carbonyldiimidazole, cyanuric chloride, terephthaloyl chloride, carbon disulfide, formaldehyde, and glutaraldehyde [Park, H.; Park, K.; Shalaby, W. S. W. Biodegradable Hydrogels for Drug Delivery, Technomic Publishing Company: Lancaster, Pa., 1993]. Crosslinking to form macroscopic hydrogels may be readily achieved using these reagents. Kabra et al. [Kabra, B. G.; Gehrke, S. H.; Spontak, R. J. Microporous, responsive hydroxypropyl cellulose gels. 1. Synthesis and microstructure. Macromolecules 1998, 31, 2166-2173] have used divinyl sulfone crosslinker to prepare macrogels of hydroxypropyl cellulose.

To prevent macrogel formation and colloidal aggregation, the polysaccharide concentration has been kept fairly low (below about 1 wt %) in the crosslinking reactions. Cai et al. [Cai, T.; Hu, Z.; Marquez, M. Synthesis and self-assembly of nearly monodisperse nanoparticles of a naturally occurring polymer. Langmuir 2004, 20, 7355-7359] have prepared nanoparticles of crosslinked hydroxypropyl cellulose using divinyl sulfone crosslinker at 0.05 wt % polymer concentration. The toxicity of divinyl sulfone is of concern in synthesizing formulations for controlled release of active agents.

The transport of small molecules such as glucose through polysaccharide hydrogels has been investigated for cell encapsulation and tissue engineering [McEntee, M.-K. E.; Bhatia, S. K.; Tao, L.; Roberts, S. C.; Bhatia, S. R. Tunable transport of glucose through ionically-crosslinked alginate gels: effect of alginate and calcium concentration. J. Appl. Polym. Sci. 2008, 107, 2956-2962]. Ionically-crosslinked alginate hydrogel beads, with an average bead diameter of 2 mm, were prepared using alginate and calcium chlorides. The researchers found a two-step release profile for glucose over a time range of 20-50 min. It should be noted that the release rates were measured by suspending the glucose-loaded spheres in pure water. The large difference in the concentration of glucose inside the sphere and the suspending fluid (pure water) resulted in a relatively rapid release of sugar (within about 50 min after suspension).

Covalent-crosslinking is expected to impart greater stability (against premature disintegration) to the hydrogel spheres, in the wide range of pH and ionic strength conditions that are encountered in the GI tract, than ionically-crosslinked hydrogels. When trisodium metaphosphate is used as the crosslinking agent, covalent-crosslinks are formed. The release rate of active agents is tuned by controlling the crosslink density of the microspheres. Notably, the release rate depends on the concentration of the active agents outside the particles, in the aqueous phase of the suspension. In some embodiments, Applicants' dispersions contain a relatively high sugar concentration in the aqueous phase. Diffusion of active agents that are e.g., nutrients, from the hydrogel microparticles typically occurs only when the nutrients get depleted from the aqueous phase. Hence, the particles act as reservoirs of nutrients such as sugar and supply nutrients within the intestinal lumen over a time period significantly beyond the duration reported in the study using ionically-crosslinked alginate beads (about 50 min) [McEntee, M.-K. E.; Bhatia, S. K.; Tao, L.; Roberts, S. C.; Bhatia, S. R. Tunable transport of glucose through ionically-crosslinked alginate gels: effect of alginate and calcium concentration. J. Appl. Polym. Sci. 2008, 107, 2956-2962]. In Applicants' formulations in embodiments where the active agents are nutrients, the nutrients dissolved in the aqueous phase will generally be initially absorbed across the intestinal ephithelium. The microparticles release entrapped nutrients at low rates initially (because of low concentration gradient), and at a faster rate when the aqueous phase nutrients are depleted (because of a greater concentration difference).

Acceptable molecular weights for polymers used in the present invention may be determined by a person of ordinary skill in the art accounting for factors such as the desired polymer degradation rate, physical properties such as mechanical strength and rate of dissolution of polymer in solvent. Typically, an acceptable range of molecular weights is of about 2,000 Da to about 2,000,000 Da, about 3,000 Da to about 1,900,000 Da, about 4,000 Da to about 1,800,000 Da, about 5,000 Da to about 1,700,000 Da, about 6,000 Da to about 1,600,000 Da, about 7,000 Da to about 1,500,000 Da, about 8,000 Da to about 1,400,000 Da, about 9,000 Da to about 1,300,000 Da, about 10,000 Da to about 1,200,000 Da, about 12,000 Da to about 1,100,000 Da, about 13,000 Da to about 1,000,000 Da, about 14,000 Da to about 900,000 Da, about 15,000 Da to about 800,000 Da, about 16,000 Da to about 700,000 Da, about 17,000 Da to about 600,000 Da, about 18,000 Da to about 500,000 Da, about 19,000 Da to about 400,000 Da, about 20,000 Da to about 300,000 Da, about 21,000 Da to about 200,000 Da, about 22,000 Da to about 100,000 Da, or about 23,000 Da to about 50,000 Da. In one embodiment, the polymer is a biodegradable polymer or copolymer.

In another embodiment, the active agent(s) can be encapsulated in microparticles or microspheres. These particles optionally comprise surfactants such as a cationic or anionic surfactant that is entrapped and fixed to the particle surface. In certain embodiments, the bioadhesive properties of the microparticles are attributed to the charged surfactants entrapped on the particle surface as the hydrophobic ends of the surfactants are embedded in the solid core and the hydrophilic ends are exposed on the surface of the microparticles.

Bioadhesive substances, also denoted mucoadhesive substances, are generally known to be materials that are capable of being bound to a biological membrane and retained on that membrane for an extended period of time. Compared with conventional controlled release systems, bioadhesive controlled release systems have the following advantages: i) a bioadhesive controlled release system localizes a biological active ingredient in a particular region, thereby improving and enhancing the bioavailability for active ingredients which may have poor bioavailability by themselves, ii) a bioadhesive controlled release system leads to a relatively strong interaction between a bioadhesive substance and a mucosa, such an interaction contributes to an increasing contact time between the controlled release system and the tissue in question and permits localization of the active released from the controlled release system to a specific site, iii) a bioadhesive controlled release system prolongs delivery of biological active ingredients in almost any non-parenteral route, iv) a bioadhesive controlled release system can be localized on a specific site with the purpose of local therapy, v) a bioadhesive controlled release system can be targeted to specific diseased tissues, and vi) a bioadhesive controlled release system is useful when conventional approaches are unsuitable, such as for certain biological active ingredients which are not adequately absorbed.

The microparticles can also include at least one co-surfactant. The co-surfactant can be a natural biologically compatible surfactant or a pharmaceutically acceptable non-natural surfactant. The co-surfactant assists in maintaining particles within the desired size range and preventing their aggregation. In certain embodiments, the co-surfactant comprises less than about 5%, less than about 4%, less than about 3%, less than about 2%, less than about 1%, less than about 0.9%, less than about 0.8%, less than about 0.7%, less than about 0.6%, less than about 0.5%, less than about 0.4%, less than about 0.3%, less than about 0.2% and less than about 0.1% by weight of the particle.

The microparticles can be formed as an aqueous continuous phase suspending a colloidal phase of submicron particles. The aqueous continuous phase of the particle suspension can contain antioxidants, preservatives, microbicides, buffers, osmoticants, cryoprotectants, and other known pharmaceutically useful additives or solutes.

The microparticles sustain the release rate of active agents for an extended period of time. For example, in some embodiments, the microparticles sustain the release of active agents, such as carbohydrates, for a period between about 1 minute and twelve hours.

The use of microparticles that provide varying rates of active agent release are contemplated. For example, the kinetics of nutrient-release may be any of the following: (i) a steady-state or zero-order release rate in which there is a substantially uniform rate of release throughout; (ii) a first-order release rate in which the rate of release declines towards zero with time; and (iii) a delayed release in which the initial rate is slow, but then increases with time.

The term “bioadhesion” relates to the attachment of a material to a biological substrate such as a biological membrane. The term “mucoadhesive substance” is in accordance with the generally accepted terminology and is used synonymously with the term “a bioadhesive substance”.

In some embodiments, a cationic surfactant is incorporated on an outer surface of the microparticle to form a bioadhesive microparticle. The surfactant is entrapped and fixed to the particle surface and fauns a coating at the interface surrounding the particle core. The interface surrounding the core is hydrophobic. The cationic surfactant also stabilizes the outer surface of the hydrophobic core component of the microparticles, thereby promoting a more uniform particle size.

Examples of surface active materials that are capable of strong bonding to the negatively charged and hydrophilic surfaces of tissues are preferable for use as cationic charged surfactants. Suitable surface active materials include straight-chain alkylammonium compounds, cyclic alkylammonium compounds, petroleum derived cationics, and polymeric cationic materials. Cetylpyridinium chloride has been found to exhibit strong bioadhesive properties on biological surfaces, and is a preferred surface active material. The surfactant is present in a proportion of about 0.01% to about 5%, about 0.05% to about 2%, by weight of the suspension. For compounds, such as certain cationic compounds, any cytotoxicity of these cationic compounds (because of their membrane disrupting ability) must be appropriately controlled.

Straight-chain alkylammonium compounds are cationic surface active materials in which one or more hydrophobic alkyl groups are linked to a cationic nitrogen atom. The linkage can also be more complex as, for example, in R—C(═O)—NHCH₂CH₂CH₂N(CH₃)₂. Alternatively, the cationic surface active material can contain more than one cationic nitrogen atom such as the class of compounds of R—NHCH₂CH₂CH₂NH₂ and derivatives thereof. Representative examples of suitable compounds for the cationic surfactant include, but are not limited to: cetyl trimethylammonium chloride (CTAB), hexadecyltrimethylammonium bromide (HDTAB), stearyl dimethylbenzylammonium chloride, lauryl dimethylbenzylammonium chloride, cetyl dimethylethylammonium halide, cetyl dimethylbenzylammonium halide, cetyl trimethylammonium halide, dodecyl ethyldimethylammonium halide, lauryl trimethylammonium halide, coconut alkyltrimethylammonium halide, and C8-C20 N,N-dialkyldimethylammonium halide.

Other suitable compounds for the cationic surfactant include, but are not limited to, bis(hydrogenated tallow alkyl) dimethylammonium chloride which is known to adsorb onto the surface with hydrophobic groups oriented away from it, 2-hydroxydodecyl-2-hydroxyethyl dimethyl ammonium chloride and N-octadecyl-N,N,N′-tris-(2-hydroxyethyl)-1,3-diaminopropane dihydrofluoride [CAS no. 6818-37-7].

Surface-active quaternary ammonium compounds in which the nitrogen atom carrying the cationic charge is part of a heterocyclic ring can be used as the cationic surfactant. Examples of suitable compounds are laurylpyridinium chloride, bromide laurylpyridinium, tetradecylpyridinium bromide, and cetylpyridinium halide where the halide is selected from chloride, bromide or fluoride.

Polymeric amines which can be used as the cationic surfactant comprise a class of polymers containing ionic groups along the backbone chain and exhibit properties of both electrolytes and polymers. These materials contain nitrogen, of primary, secondary, tertiary or quaternary functionality in their backbone and may have weight average molecular weights as low as about 100 Da or higher than about 100,000 Da. Suitable polymeric amines useful as a cationic surfactant include, but are not limited to, polydimeryl polyamine available from General Mills Chemical Co., polyamide, polyacrylamides, polydiallyldimethylammonium chloride, polyhexamethylene biguanide compounds, and also other biguanides, for example those disclosed in U.S. Pat. Nos. 2,684,924, 2,990,425, 3,183,230, 3,468,898, 4,022,834, 4,053,636 and 4,198,425, herein incorporated by reference into this application, 1,5-dimethyl-1,5-diazaundecamethylene polymethobromide, such as “Polybrene” manufactured by Aldrich, polyvinylpyrrolidone and their derivatives, polypeptides, poly(allylamine) hydrochloride, polyoxyethylenated amines, and polyethyleneimine, such as “Polymin” manufactured by BASF.

Suitable polymeric materials for the cationic surfactant also include surface active cationic polymers prepared by converting a fraction of the amino groups to their acyl derivatives. For example, the polyethyleneimine is first condensed with less than the stoichiometric quantity of acid halides thus alkylating some of the amino groups and the remaining amino groups are then condensed with hydrogen halides such as hydrogen chloride or, preferably, hydrogen fluoride. The surface activity of these compounds varies with the number of amino groups which are acylated and with the chain length of the acylating group R—C(═O). The condensation reaction can be performed with stearic or oleic acid chlorides in the presence of a solvent containing metal fluoride, such as silver fluoride, in such a manner that metal chloride formed in the reaction precipitates from the solvent.

Also suitable, for the purpose of this invention, are cationic derivatives of polysaccharides such as dextran, starch or cellulose, for example, diethylaminoethyl cellulose. Examples of applicable copolymers based on acrylamide and a cationic monomer are available from Hercules Inc. under the trade name RETEN including RETEN 220, or from National Adhesives under the trade name FLOC AID including FLOC AID 305. Other useful acrylamide-based polyelectrolytes are available from Allied Colloids under the trade name PERCOL. Further examples of suitable materials are cationic guar derivatives such as those sold under the trade name JAGUAR by Celanese-Hall.

In another embodiment, the microparticles comprise a hydrophobic core that is formed of a biodegradable hydrophobic material having barrier properties. Suitable, nontoxic, pharmaceutical solid core materials are inert hydrophobic biocompatible materials with a melting range between about 50° C. and about 120° C., between about 60° C. and about 110° C., between about 70° C. and about 100° C. or between about 80° C. and about 90° C. Examples include, but are not limited to, natural, regenerated, or synthetic waxes including: animal waxes, such as beeswax; lanolin and shellac wax; vegetable waxes such as carnauba, candelilla, sugar cane, rice bran, and bayberry wax; mineral waxes such as petroleum waxes including paraffin and microcrystalline wax; cholesterol; fatty acid esters such as ethyl stearate, isopropyl myristate, and isopropyl palmitate; high molecular weight fatty alcohols such as cetostearyl alcohol, cetyl alcohol, stearyl alcohol, and oleyl alcohol; solid hydrogenated castor and vegetable oils; hard paraffins; hard fats; biodegradable polymers such as polycaprolactone, polyamides, polyanhydrides, polycarbonates, polyorthoesters, polylactic acids, and copolymers of lactic acid and glycolic acid; cellulose derivatives and mixtures thereof. Other hydrophobic compounds which may be used in the present invention include triglycerides, preferably of food grade purity or better, which may be produced by synthesis or by isolation from natural sources. Natural sources may include animal fat or vegetable oil, such as soy oil, a source of long chain triglycerides (LCT). Other suitable triglycerides are composed predominantly of medium length fatty acids (C10-C18), denoted medium chain triglycerides (MCT). The fatty acid moieties of such triglycerides can be unsaturated, monounsaturated or polyunsaturated. Mixtures of triglycerides having various fatty acid moieties are also useful for the present invention. In embodiments comprising a core, the core can comprise a single hydrophobic compound or a mixture of hydrophobic compounds. Hydrophobic materials are known to those skilled in the art and are commercially available, as described in the list of suitable carrier materials in Martindale, The Extra Pharmacopoeia, 28^{th} ed.; The Pharmaceutical Press: London, 1982; pp 1063-1072. Considerations in the selection of the core material include good barrier properties to the active ingredients and sensory markers, low toxicity and irritancy, biocompatibility, stability, and high loading capacity for the active ingredients of interest.

An amphiphilic or nonionic co-surfactant can be used in the microparticles of the present invention to provide improved stability. Co-surfactants can be formed of natural compounds or nonnatural compounds. Examples of natural compounds are phospholipids and cholates. Examples of nonnatural compounds include: polysorbates, which are fatty acid esters of polyethoxylated sorbitol sold by Unigema surfactants as Tween; polyethylene glycol esters of fatty acids from sources such as castor oil; polyethoxylated fatty acid, such as stearic acid; polyethoxylated isooctylphenol/formaldehyde polymer; poloxamers, such as, poly(oxyethylene)poly(oxypropylene) block copolymers available from BASF as Pluronic; polyoxyethylene fatty alcohol ethers available from ICI surfactants as Brij; polyoxyethylene nonylphenyl ethers sold by Union Carbide as Triton N; polyoxyethylene isooctylphenyl ethers sold by Union Carbide as Triton X; and SDS. Mixtures of surfactant molecules, including mixtures of surfactants of different chemical types, can be used in the present invention. Surfactants preferably are suitable for pharmaceutical administration and compatible with the drug to be delivered.

Particularly suitable surfactants include phospholipids, which are highly biocompatible. Examples of suitable phospholipids are phosphatidylcholines (lecithins), such as soy or egg lecithin. Other suitable phospholipids include phosphatidylglycerol, phosphatidylinositol, phosphatidylserine, phosphatidic acid, cardiolipin, and phosphatidylethanolamine. The phospholipids may be isolated from natural sources or prepared by synthesis. Phospholipid surfactants are believed to usually form a single monolayer coating of the hydrophobic core. The co-surfactant can be present in an amount less than about 5%, less than about 1%, and less than about 0.1%, relative to the weight of hydrophobic core component. In some embodiments, one or more co-surfactants can be used.

In another embodiment, the active agents comprise compounds that modulate uptake of carbohydrates. For example, in the gastrointestinal tract, chromium and vanadium (either individually, or in concert) modulate sugar transport (e.g., glucose transport) by typically slowing glucose absorption. Slower glucose absorption slows insulin release and reduces excessive insulin responses in response to rising blood glucose levels after a meal. This benefits pancreatic secretion of insulin by reducing both the glucose load and rate of glucose load over the initial phases of glucose detection, absorption and metabolism by the body. Reduced rates of glucose loading reduce the stress on beta cells normally associated with the insulin response to rising glucose. Moreover, slower or modulated glucose absorption permits more time for insulin to stimulate normal sugar metabolic routes either before glucose loading is complete, or during a slower rate of glucose loading. Consequently, insulin dependent mechanisms have more time to prepare for the arrival of sugars from the intestine. This modulation of glucose absorption improves short-term insulin modulation in the liver, muscle, and adipose tissue. These effects in the gastrointestinal tract are, in all likelihood, short-term responses, and they are not necessarily associated with the longer-term systemic effects of chromium and vanadium administration.

In addition, chromium and vanadium may potentially slow glucose metabolism by interacting with the intestine, particularly the epithelium of the intestine responsible for sugar metabolism (including absorption). One primary mechanism for sugar transport in the gut is sodium facilitated sugar transport. Such transporters are located in the lumenal membrane of the epithelium. The basolateral membrane may also have an additional sugar transporter that facilitates transport out the cell and into the blood. For net sugar absorption from the lumen of the gut to the blood, sodium facilitated sugar transport generally requires a sodium concentration favorable to the diffusion of sodium into the epithelium cell from the lumen. This concentration gradient is largely generated by the active transport of the Na/K ATPase in the epithelium cells, which generally transports three sodium atoms out of the cell to the blood side of the epithelium in exchange for two sodium atoms in the reverse direction.

Each cycle of the pump involves hydrolysis of one ATP to transport sodium and potassium against their respective concentration gradients. The hydrolysis reaction involves a divalent cation, typically magnesium. In many instances, however, other divalent cations may substitute or enter into the hydrolysis reaction with varying degrees of catalytic activity or inhibition. Substitution of trivalent cations for divalent cations in the cycle generally leads to significant inhibition of the pumping activity and/or dephosphorylation from the phosphoenzyme intermediate state. Chromium may thus inhibit the Na/K ATPase activity by substituting for magnesium and thereby inhibiting, relative to magnesium, catalytic and transport activity, giving rise to a decreased sodium gradient across the lumenal membrane. The reduced gradient effects sugar transport by reducing the thermodynamic and kinetic forces favoring sugar entry from the gut.

In addition, during the hydrolysis of ATP in the catalytic cycle of the Na/K ATPase, a phosphoenzyme intermediate (EP) is formed between phosphate and an aspartic acid at the active site of APTase. This covalent EP is transient and is chemically distinct from phosphorylated proteins associated with kinases and phosphatases, which have also been shown to be affected by vanadium. Formation of EP in the catalytic cycle for Na/K ATPase is inhibited by vanadate present at low concentrations of less than 1 micromolar. Vanadate binds to the active site as a transition state analog of phosphate in a vanadyl-enzyme, or EV complex, rather than EP. The EV complex is highly stable, as the kinetics of loss of vanadate from the EV complex is relatively slow. Vanadate may thus effectively inhibit the Na/K ATPase by disrupting catalysis, through the formation of EV, giving rise to a decreased sodium gradient across the lumenal membrane. Consequently, the reduced gradient reduces sugar entry from the intestine.

Chromium and vanadium also operate at the systemic level after absorption of the two transition metals from the gut. Major sites of activity include the liver, muscle, and adipose tissue. Vanadium may have particular activity with respect to phosphorylation systems, including the many phosphorylated proteins responsible for modulating metabolism. Chromium may also modulate metabolism at the cellular level. These systemic effects generally improve the action of insulin and/or metabolic pathways associated with sugar and/or lipid metabolism.

In regard to absorption and metabolism of the subject compositions, and the different components thereof, features of the alimentary tract may affect how compositions of the present invention, and methods of using the same, are utilized when ingested orally. The elements of the alimentary tract, including the gastrointestinal tract, may affect the dosage required for any such modality. Such features are well known to those of ordinary skill in the art.

In another embodiment, the active agent compositions are formulated into unit dosage forms such as tablets, caplets, powder, granules, beads, chewable lozenges, capsules, liquids, aqueous suspensions or solutions or similar dosage fowls, using conventional equipment and techniques known in the art. Such formulations typically include a solid, semisolid, or liquid carrier. Exemplary carriers include lactose, dextrose, sucrose, sorbitol, mannitol, sutarches, gum acacia, calcium phosphate, mineral oil, cocoa butter, oil of theobroma, alginates, tragacanth, gelatin syrup, methyl cellulose, polyoxyethylene sorbitan monolaurate, methyl hydroxybenzoate, propyl hydroxybenzoate, talc, magnesium stearate, and the like.

Other formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of an active agent or components thereof as an active ingredient. An active agent or components thereof may also be administered as a bolus, electuary, or paste.

In other formulations, the active agents are provided in beverages. The beverages of this invention can be carbonated beverages e.g., flavored seltzer waters, soft drinks, or mineral drinks, as well as non-carbonated juices, punches and concentrated forms of these beverages. Beverages, especially juice and cola beverages, which are carbonated in the manner of soft drinks, as well as “still” beverages and nectars and full-strength beverages or beverage concentrates that contain at least about 45% by weight of juice are also contemplated.

By way of example, the fruit juices and fruit flavors used herein include grape, pear, passion fruit, pineapple, banana or banana puree, apricot, orange, lemon, grapefruit, apple, cranberry, tomato, mango, papaya, lime, tangerine, cherry, raspberry, carrot and mixtures thereof. Additionally, artificial flavors, e.g., cola, or natural flavors derived from these juices can be used in the beverages. Chocolate flavors and other non-fruit flavors can also be used to make beverages containing the active agent, for example, vitamin and mineral supplements. Additionally, milk, obtained from cows or synthetic, is a contemplated beverage to which the powder compositions of this invention can be added. The milk may itself include other beverage components, in particular flavors such as chocolate, coffee, or strawberry. As used herein, the term “juice product” refers to both fruit and vegetable juice beverages and fruit and vegetable juice concentrates which comprise at least about 45% fruit juice. Vegetable when used herein includes both nonfruit edible plant parts such as tubers, leaves, rinds, and also if not otherwise indicated, any grains, nuts, beans, and sprouts which are provided as juices or beverage flavorings.

In one embodiment, sport beverages can be supplemented by the powder compositions of the present invention. Typical sport beverages contain water, sucrose syrup, glucose-fructose syrup, and natural or artificial flavors. These beverages can also contain citric acid, sodium citrate, monopotassium phosphate, as well as other materials that are useful in replenishing electrolytes lost during perspiration.

As used herein, the term “juice beverage” refers to a fruit or vegetable juice product that is in a single-strength, ready-to-serve, drinkable form. Juice beverages of the present invention can be of the “full-strength” type that typically comprise at least about 95% juice. Full strength juice beverages also include those products of 100% juice such as, for example, orange, lemon, apple, raspberry, cherry, apricot, pear, grapefruit, grape, lime, tangerine, carrot, pineapple, melon, mango, papaya, passion fruit, banana and banana puree, cranberry, tomato, carrot, cabbage, celery, cucumber, spinach, and various mixtures thereof. Juice beverages also include extended juice products which are referred to as “nectars.” These extended juice products typically comprise from about 50% to about 90%, about 55% to about 85%, about 60% to about 80%, about 65% to about 75% juice, from about 50% to about 70% juice. Nectars usually have added sugars or artificial sweeteners or carbohydrate substitutes. As used herein, the term “citrus juice” refers to fruit juices selected from orange juice, lemon juice, lime juice, grapefruit juice, tangerine juice and mixtures thereof.

As used herein, the term “juice materials” refers to concentrated fruit or vegetable juice, plus other juice materials such as juice aroma and flavor volatiles, peel oils, and pulp or pomace. As used herein, the term “juice concentrate” refers to a fruit or vegetable juice product which, when diluted with the appropriate amount of water, forms drinkable juice beverages. Juice concentrates within the scope of the present invention are typically formulated to provide drinkable beverages when diluted with 3 to 5 parts by weight water.

As used herein the term “beverage concentrate” or “bottling syrup” refers to a mixture of flavors, water, and from about 10% to about 60%, about 20% to about 50% or about 30% to about 40% sugar or carbohydrate substitute, e.g., sucrose, dextrose, corn syrup solids, fructose, dextrins, polydextrose and mixtures thereof.

The flavor component of the beverages and beverage concentrates contains flavors selected from fruit flavors, vegetable flavors, botanical flavors, and mixtures thereof. As used herein, the term “fruit flavor” refers to those flavors derived from the edible reproductive part of a seed plant, especially one having a sweet pulp associated with the seed, and “vegetable flavor” refers to flavors derived from other edible parts of seed and other plants. Also included within the term “fruit flavor” and “vegetable flavor” are synthetically prepared flavors made to simulate fruit or vegetable flavors derived from natural sources. Particularly preferred fruit flavors are the citrus flavors including orange, lemon, lime and grapefruit flavors. Besides citrus flavors, a variety of other fruit flavors can be used such as apple, grape, cherry, pineapple, mango and papaya flavors and the like. These fruit flavors can be derived from natural sources such as juices and flavor oils, or can be synthetically prepared. As used herein, the term “botanical flavor” refers to flavors derived from parts of a plant other than the fruit; i.e., derived from nuts, bark, roots and leaves, and beans such as coffee, cocoa, and vanilla. Also included within the term “botanical flavor” are synthetically prepared flavors made to simulate botanical flavors derived from natural sources. Examples of such flavors include cola, tea, coffee, chocolate, vanilla, almond, and the like. Botanical flavors can be derived from natural sources such as essential oils and extracts, or can be synthetically prepared.

The flavor component can comprise a blend of various flavors, e.g., lemon and lime flavors, cola flavors and citrus flavors to form cola flavors, etc. If desired, juices such as orange, lemon, lime, apple, grape, carrot, celery, and like juices can be used in the flavor component. The flavors in the flavor component are sometimes formed into emulsion droplets that are then dispersed in the beverage concentrate. Because these droplets usually have a specific gravity less than that of water and would therefore form a separate phase, weighting agents (which can also act as clouding agents) are typically used to keep the emulsion droplets dispersed in the beverage. Examples of such weighting agents are brominated vegetable oils (BVO) and rosin esters, in particular the ester gums. See Green, L. F. Developments in Soft Drinks Technology; Applied Science Publishers: London, 1978; Vol. 1, pp 87-93, for a further description of the use of weighting and clouding agents in liquid beverages. Besides weighting agents, emulsifiers and emulsion stabilizers can be used to stabilize the emulsion droplets. Examples of such emulsifiers and emulsion stabilizers include the gums, pectins, celluloses, polysorbates, sorbitan esters and propylene glycol alginates. See Green, L. F. supra at p. 92. The particular amount of the flavor component effective for imparting flavor characteristics to the beverages and beverage concentrates (“flavor enhancing”) can depend upon the flavor(s) selected, the flavor impression desired, and the form of the flavor component.

The flavor component can comprise at least 0.05% by weight of the beverage composition, and typically from 0.1% to 2% by weight for carbonated beverages. When juices are used as the flavor, the flavor component can comprise, on a single-strength basis, up to 25% fruit juice by weight of the beverage, including for example, from 5% to 15% juice by weight for carbonated beverages.

Carbon dioxide can be introduced into the water that is mixed with the beverage syrup or into the drinkable beverage after dilution to achieve carbonation. The carbonated beverage can be placed into a container such as a bottle or can and then sealed. Any conventional carbonation methodology can be used to make the carbonated beverages of this invention. The amount of carbon dioxide introduced into the beverage will depend upon the particular flavor system used and the amount of carbonation desired. Usually, carbonated beverages of the present invention contain from 1.0 to 4.5 volumes of carbon dioxide. In certain embodiments, the carbonated beverages contain from 2 to about 3.5 volumes of carbon dioxide.

The present invention is also particularly suited for the supplementation of beverages and beverage concentrates, including water and citrus juices. The beverages can contain from 3% to 100% juice or from about 0.05% to about 10% of an artificial or natural flavor, such as orange juice. The concentrated orange juice, orange juice aroma and flavor volatiles, pulp and peel oils used in the method of the present invention can be obtained from standard orange juice. See Nagy, S.; Shaw, P. E.; Veldhuis, M. K. Citrus Science and Technology; AVI Publishing: Westport, Conn., 1977; Vol. 2, pp 177-252 for standard processing of oranges, grapefruit, and tangerines. (See also Nelson et al. Fruit and Vegetable Juice Processing Technology, 3rd ed.; AVI Publishing: Westport, Conn., 1980; pp. 180-505, for standard processing of noncitrus juices such as apple, grape, pineapple, etc. to provide sources of juice and juice materials for noncitrus juice products).

Juices from different sources are frequently blended to adjust the sugar to acid ratio of the juice. Different varieties of oranges can be blended or different juices can be blended to get the desired flavor and sugar to acid ratio. A sugar to acid ratio of from about 8:1 to about 20:1 is considered acceptable for fruit juices. Sugar to acid ratios are typically from about 11:1 to about 15:1, especially for citrus juices. Sweeteners include the sugars normally present in juice products, for example glucose, sucrose, and fructose. Sugars also include high fructose corn syrup, invert syrup, sugar alcohols, including sorbitol, refiners syrup, and mixtures thereof. In addition to sugar, extended juice beverages of the present invention can contain other sweeteners. Other suitable sweeteners include saccharin, cyclamates, acetosulfam, L-aspartyl-L-phenylalanine lower alkyl ester sweeteners (e.g., aspartame). One sweetener for use in such extended juice products is aspartame. For single-strength juice beverages, the sugar content can range from about 2° to about 16° Brix (16° Brix means the juice contains about 16% soluble solid, and so on). Typically, the sugar content of such beverages depends upon the amount of juice contained herein.

In solid dosage forms for oral administration (e.g., capsules, tablets, pills, dragees, powders, granules, and the like), the active agent or components thereof is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In some embodiments, in the case of capsules, tablets, and pills, for example, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the active agent or components thereof moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Tablets and other solid dosage forms may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which may be dissolved in sterile water, or sonic other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which may be used include polymeric substances and waxes. The active ingredient may also be in micro-encapsulated form, if appropriate.

Liquid dosage forms for oral administration include pharmaceutically acceptable emulsions, microemulsions, solutions, suspensions, syrups, and elixirs. In addition to the active agent or component, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions may also include adjuvants such as wetting agents, emulsifying and suspending agents, sweetening, flavoring, coloring, perfuming and preservative agents. Suspensions, in addition to the active agent or components thereof, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

A composition of the invention can be administered as a capsule or tablet containing a single or divided dose of the active agent. Preferably, the composition is administered as a sterile solution, suspension, or emulsion, in a single or divided dose. Tablets may contain carriers such as lactose and corn starch, and/or lubricating agents such as magnesium stearate. Capsules may contain diluents including lactose and dried corn starch.

A tablet may be made by compressing or molding the active ingredient optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Molded tablets may be made by molding in a suitable machine, a mixture of the powdered active ingredient and a suitable carrier moistened with an inert liquid diluent.

When preparing dosage forms incorporating the compositions of the invention, the compounds may also be blended with conventional excipients such as binders, including gelatin, pregelatinized starch, and the like; lubricants, such as hydrogenated vegetable oil, sutearic acid, and the like; diluents, such as lactose, mannose, and sucrose; disintegrants, such as carboxymethylcellulose and sodium starch glycolate; suspending agents, such as povidone, polyvinyl alcohol, and the like; absorbants, such as silicon dioxide; preservatives, such as methylparaben, propylparaben, and sodium benzoate; surfactants, such as sodium lauryl sulfate, polysorbate 80, and the like; colorants such as F.D. & C. dyes and lakes; flavorants; and sweeteners.

In certain embodiments, where the active agent is, for example, glucose, the “dose” of glucose can be calculated to be delivered according to the methods of the invention so as to optimize the effect of the glucose. To do this, it is desirable to consider the basal glucose level, the bioavailability of glucose, the elimination rate of glucose (i.e., consumption rate), blood flow to the target organ (e.g., the brain), and the volume of the body's plasma. For example, the calculations below assume a 25% reduction in blood flow to the cerebral circulation (i.e., 25% ischemia), and thus, a 25% decrease in delivery of glucose to the brain. In the example in Table 2 below, a 25% increase in glucose delivery would be needed to overcome the reduction in delivery of glucose to the brain because of ischemia (since blood flow to the brain cannot be adjusted, one can simply increase the amount of glucose in the blood). Thus, assuming a 100% bioavailability of glucose and a constant rate of elimination of glucose (i.e., no change in energy consumption, such as through exercise), then a dose of 2.45 gm of glucose would be needed to increase the plasma glucose by 17.5 mg/dL (assumes a plasma volume of 14 L in a typical human).

In certain embodiments, the proposed approach is to engineer controlled release of digestible carbohydrates from an aqueous dispersion of suitable micro- or nanospheres. Important digestible carbohydrates include: the monosaccharides glucose, fructose, and galactose; the dissacharides trehalose, sucrose, maltose, and lactose; and the polysaccharide, starch. Starch is broken down into dextrins by salivary amylase (in the mouth) and pancreatic amylase (in the small intestine). Dextrin is acted upon by the brush border enzymes in the small intestine, which also convert the double sugars into simple sugars. The monosaccharides are finally transported across the intestinal epithelium into the bloodstream. In particular embodiments, the instant methods provide for controlled release of digestible carbohydrates, especially the simple sugars, glucose and fructose, for sustained uptake into the blood.

A basic understanding of the physiology of the gastrointestinal (GI) tract is useful in the design of the delivery system. The retention time of food in the stomach is up to 2 hours and depends, among other factors, on the calorific value of the meal (see, e.g., Hadi, N. A.; Giouvanoudi, A.; Morton, R.; Horton, P. W.; Spyrou, N. M. Variations in gastric emptying times of three stomach regions for simple and complex meals using scintigraphy. IEEE Transactions on Nuclear Science 2002, 49, 2328-2331). The controlled release system should be able to withstand the acidic pH (1-3) of the stomach during gastric retention, without releasing the sugar payload. Residence time in the small intestine, where most of the nutrient absorption occurs, is about 3 h. For nutrient delivery over a longer time period, it is typically necessary to prolong intestinal retention which may be achieved by encapsulating the nutrient in a carrier with mucoadhesive properties. Hydrophilic polymers containing carboxylic acid groups exhibit good mucoadhesive properties. With respect to controlled release systems for sugar, a key step in the design of such a system is the selection of a carrier material for encapsulating carbohydrates. Polysaccharides and their derivatives are polymers of choice as carriers for sustained-release drug delivery and scaffolds in tissue engineering because of their non-toxic nature and excellent biocompatibility (see, e.g., Dumitriu, S.; Dumitriu, M. Hydrogels as support for drug delivery systems. In Polysaccharides in Medicinal Applications; Dumitriu, S. Ed.; Dekker: New York, 1996; pp 705-764; Coviello, T.; Matricardi, P.; Marianecci, C.; Alhaique, F. Polysaccharide hydrogels for modified release formulations. J. Control. Rd. 2007, 119, 5-24 and Kong, H.; Mooney, D. J. Polysaccharide-based hydrogels in tissue engineering. In Polysaccharides, 2.sup.nd ed.; Dumitriu, S., Ed.; Dekker: New York, 2005; pp 817-837). They have also been used for flavor encapsulation in food formulations (see, e.g., Madene, A.; Jacquot, M.; Scher, J.; Desobry, S. Flavour encapsulation and controlled release—a review. International Journal of Food Science and Technology 2006, 41, 1-21).

Blends of polysaccharides can be used to synthesize aqueous dispersions of micro- or nanoparticles. Hydrophobically modified polysaccharides such as hydroxypropyl cellulose or hydroxyethyl cellulose are known to spontaneously form nanoparticles in water. Interpenetrating polymer networks of these polymers, with polysaccharides containing carboxylic acid groups, are synthesized. The monomeric unit of the carboxymethylcellulose backbone, for example, consists of D glucose residues linked through β-(1→4) bonds. Alginates are composed of (1→4)-linked β-D-mannuronic acid and a-L-guluronic acid monomers that vary in amount and sequential distribution along the polymer chain depending on the source of alginate. Hyaluronic acid is a straight polymer consisting of alternating (1→4)-linked 2-acetamide-2-deoxy-β-D-glucose and (1→3) linked β-D-glucuronic acid.

In some embodiments, to increase stability of the particles in the GI tract, the particles are crosslinked to form hydrogels. Different crosslinking mechanisms can be employed to achieve the desired release kinetics. Crosslinking is performed using free radical initiators such as persulfate salts, or redox systems involving ascorbic acid, or a naturally occurring crosslinker such as genipin. Ionic crosslinking can also be performed. Anionic polysaccharides such as gellan can be used for ionic crosslinking, instead of chemicals such as borax, which may not be desirable in a food formulation.

It is expected that the carboxy containing hydrogel particles are in a collapsed state in the acidic environment of the stomach. Hence, the encapsulated one or more active agents are retained within the particles in the stomach. The hydrogel particles will achieve an expanded state when they reach the small intestine (pH 5-7), and will release the encapsulated one or more active agents at a rate faster than that in the stomach.

Several researchers have investigated the synthesis of polysaccharide particles and hydrogels for controlled release. Most of these studies were, however, focused on incorporating relatively hydrophobic drugs or protein macromolecules in the carriers. An objective of the instant invention is to encapsulate small hydrophilic molecules such as sugars. The equilibrium partitioning of sugar molecules between the hydrogel particles and the aqueous phase is determined. Due to similarities in the chemical structures of the polysaccharide carrier and the encapsulated monosaccharides, it is expected that the encapsulation efficiency of polysaccharide hydrogels are higher than those of other hydrogels.

There are only a few studies that have reported delayed release systems for carbohydrates. Fox and Allen (Fox, G. J.; Darlene, A. Method and composition for controlling the release of carbohydrates by encapsulation. U.S. Pat. No. 5,536,156, Jul. 16, 1996) have coated carbohydrate microparticles with an edible delayed-release coating. The coated carbohydrate, when orally ingested, causes a time delayed release of the carbohydrate into the digestive system. The coated particles were 30 to 100 .mu.m in size and were stored in solid particulate form. In contrast, Applicants seek to develop controlled release particles that are dispersed in an aqueous medium. Lake and Smith (Lake, M.; Smith, U. Composition and method for long-term glycemic control. Int. Pat. Appl. WO/2006/022585, Feb. 3, 2006) have reported the preparation of starch granules that can be used for improved long-term control of blood glucose in a diabetic patient. The delayed-release starch formulation was designed to reduce the incidence of nocturnal hypoglycemia, wherein the patient would ingest a therapeutic amount of starch granules at bedtime. Zecher (Zecher, D. C. Controlled release carbohydrate embedded in a crosslinked polysaccharide. Int. Pat. Appl. WO/2000/032064, Aug. 6, 2000) has reported a similar controlled release carbohydrate composition consisting of covalently crosslinked polysaccharides. However, the crosslinked carbohydrates were not in a particulate form, and were not in the folio of aqueous suspensions.

The following sections will describe methods for the synthesis of polysaccharide hydrogels.

Hydrophobized polysaccharides are highly promising in the synthesis of nanoparticles because of their self-assembling properties in aqueous environment. Akiyoshi and Sunamoto (Akiyoshi, K.; Sunamoto, J. Supramolecular assembly of hydrophobized polysaccharides. Supramolecular Science 1996, 3, 157-163) found that polysaccharides that were functionalized with hydrophobes such as cholesterol spontaneously formed nanoparticles when dispersed in water. The size, density, and colloidal stability of the nanoparticle could be controlled by tailoring the grafting density and degree of hydrophobicity of the hydrophobe. Polysaccharides such as pullulan, dextran, and mannan were partly substituted by various hydrophobic groups such as long alkyl chains and cholesterol. For example, pullulan with a molecular weight of 55 kDa, when functionalized with cholesterol (.about.1.7 cholesterol moieties per 100 units of glucose) spontaneously formed nanoparticles that were 20-30 nm in size (Akiyoshi, K.; Deguchi, S.; Tajima, H.; Nishikawa, T.; Sunamota, J. Self-assembly of hydrophobized polysaccharide: Structure of hydrogel nanoparticle and complexation with organic compounds. Proc. Japan Acad. 1995, 71, 15-19). The cholesterol bearing pullulan self-aggregated to form monodisperse stable nanoparticles after ultrasonification of the suspension in water. No coagulation occurred even after heating at 90° C. for 1 h. These nanoparticles were used for hosting hydrophobic substances such as antitumor adriamycin (Akiyoshi, K.; Taniguchi, I.; Fukui, H.; Sunamoto, J. Hydrogel nanoparticle formed by self-assembly of hydrophobized polysaccharide. Stabilization of adriamycin by complexation. European Journal of Pharmaceutics and Biopharmaceutics 1996, 42, 286-290) and various water-soluble proteins, but encapsulation of small water-soluble molecules was not reported.

Chakraborty et al. (Chakraborty, S.; Sahoo, B.; Teraoka, I.; Gross, R. A. Solution properties of starch nanoparticles in water and DMSO as studied by dynamic light scattering. Carbohydrate Polymers 2005, 60, 475-481) have studied the solution properties of starch nanoparticles in water using dynamic light scattering. The nanoparticles were obtained from Ecosynthetix (Lansing, Mich.), and were synthesized from corn starch using glyoxal as crosslinker. A mixture of starch, glycerol (18 wt % of dry starch), and glyoxal (0.1-10 wt %) was extruded to obtain crosslinked starch granules. The granules were cryogenically ground and sieved to obtain particles smaller than 150 nm in diameter. Dynamic light scattering or the particles in water indicated two main populations, with mean diameters of 40 and 300 nm, consisting of isolated starch nanoparticles and their aggregates, respectively. At higher concentration (about 3% w/w), a third peak appeared at around 1 μm, because of particle aggregation. Control of particle aggregation is an important step in the design of carbohydrate nanoparticles.

A key feature of the instant polysaccharide hydrogels is their pH responsiveness. Ideally, the hydrogels should not swell in the acidic environment of the stomach, but should swell upon entry into the small intestine and release the encapsulated sugars at a controlled rate. This section reviews an extreme case where the polysaccharide matrix was insoluble in acidic environments, while it completely dissolved at higher pH values.

Scleroglucan is a branched homopolysaccharide that gives only D-glucose upon complete hydrolysis. The polymer consists of a main chain of (1→3)-linked β-D-glucopyranosyl units. At every third unit along the main chain, the polymer bears a single (1→6)-linked β-D-glucopyranosyl unit as a branch. The glucopyranose side chain of scleroglucan was oxidized by means of a two-step reaction: first with periodate, to Ryan an aldehyde derivative, and then with chlorite, which resulted in the carboxylated derivative called sclerox (see, e.g., Coviello, T.; Palleschi, A.; Grassi, M.; Matricardi, P.; Bocchinfuso, G.; Alhaique, F. Scleroglucan: A versatile polysaccharide for modified drug delivery. Molecules 2005, 10, 6-33). By varying the ratio between oxidizing agent and polysaccharide, the polymer could be oxidized to a different extent. It was found that above a 60% oxidation, sclerox became sensitive to environmental conditions giving a reversible sol-gel transition mediated by pH. Permeation of model molecules occurred at different rates through the sol and the gel, and consequently, release from sclerox tablets showed different profiles in the two environments simulating the gastric and the intestinal fluids, respectively.

The formulation viscosity is expected to increase with an increase in particle concentration. As a first approximation, viscosity of a suspension is related to the particle concentration through the Einstein's equation, η=η_{w} (1+2.5φ), where η is the viscosity of the dispersion, η_{w} is the viscosity of the aqueous phase, and φ is the volume fraction of particles in the dispersion. The particle volume fraction is given by

$$φ = {[ 1 + ( \frac{{ρ}_{p}}{{ρ}_{w}} ) \cdot ( \frac{1}{m} - 1 ) ]}^{- 1} ,$$

where ρₚ is the density of the particles, ρ_{w} is the aqueous phase density, and m is the mass fraction of particles in the dispersion. Dispersion viscosity also depends on the interparticle distance, H, which is the average distance between the surfaces of two neighboring particles in the dispersion. For a population of monodisperse particles with hexagonal close packed structure, the interparticle distance is given by

$$H = D \{ {( \frac{0.74}{φ} )}^{1 / 3} - 1 \}$$

where D is the particle diameter. Therefore, for a given mass fraction of polymer in the dispersion (that is, a fixed φ) the dispersion viscosity is expected to be higher when the particles are smaller in size. In this Example, the viscosity of the dispersion is tailored to be close to that of water (about 1 mPas).

Selection of a suitable crosslinker is a key step in the preparation of polysaccharide hydrogels for food formulations. Clearly, toxicity of the crosslinking chemical precludes its use. Genipin is a naturally occurring crosslinker for proteins and polysaccharides, and is obtained from gardenia fruit extracts. It has attracted significant interest in the synthesis of polysaccharide hydrogels. It has low acute toxicity (LD₅₀ i.v. 382 mg/kg in mice) and is much less toxic than most other chemical crosslinking agents such as glutaraldehyde.

Alternatively, crosslinking can be achieved using free radicals. Free radical initiators such as ammonium persulfate are listed in GRAS list of chemicals, and can be used in food formulations.

Gellan can also be used as an ionic crosslinking agent. Gellan is an anionic microbial polysaccharide that is well known for its gelling properties in the presence of counterions, especially divalent ions, like calcium. Gellan has been used as a crosslinker for scleroglucan.

Carrageenans are linear sulfated biopolymers, composed of D-galactose and 3,6-anhydro-D-galactose units. κ-Carrageenan beads are prepared by gelling with monovalent ions (often K⁺) and sometimes divalent ions. Alginates are linear polysaccharides produced by algae, which contain varying amounts of (1→4)-linked β-D-mannuronic acid and .alpha.-L-guluronic acid residues. Mohamadnia et al. have synthesized ionically crosslinked beads of carbohydrate biopolymers. κ-carrageenan and sodium alginate (see, e.g., Mohamadnia, Z.; Zohuriaan-Mehr, M. J.; Kabiri, K.; Jamshidi, A.; Mobedi, H. pH-Sensitive IPN hydrogel beads of carrageenan-alginate for controlled drug delivery. J. Bioactive Compat. Polym. 2007, 22, 342-356 and Mohamadnia, Z.; Zohuriaan-Mehr, M. J.; Kabiri, K.; Jamshidi, A.; Mobedi, H. Ionically crosslinked carrageenan-alginate hydrogel beads. Journal of Biomaterials Science: Polymer Edition 2008, 19, 47-59). Alginate gelation takes place when divalent or trivalent cations (usually Ca.sup.2+) interact ionically with guluronic acid residues, resulting in the formation of a three-dimensional network. Alginate-Ca²⁺ hydrogels have been studied for controlled release oral drug formulations (see, e.g., Bajpai, S. K.; Sharma, S Investigation of swelling/degradation behavior of alginate beads crosslinked with Ca²⁺ and Ba.²⁺ ions. React. Func. Polym. 2004, 59, 129-140).

In certain embodiments, a blend of hydrophobically modified polysaccharide such as hydropropyl cellulose, methyl cellulose, ethyl cellulose, hydroxypropyl methyl cellulose, hydroxyethyl methyl cellulose, ethyl hydroxyethyl cellulose, methyl ethyl hydroxyethyl cellulose, hydroxyethyl cellulose, and/or cellulose acetate and a carboxy containing polysaccharide such as alginate or carboxymethyl cellulose is used to prepare the hydrogel particles. The hydrophobically modified polysaccharide results in spontaneous particle formation due to phase separation in water, while the polysaccharide containing carboxylic acid groups imparts a pH-responsive behavior and will also increase intestinal transit time. A review of the formation of hydrogels (both macroscopic gels and aqueous dispersions) using a blend of polysaccharides follows.

Ichikawa et al. have synthesized nanoparticle suspensions of 0.5 wt % concentration by self-assembly of chitosan (with a degree of deacetylation about 77%) and carboxymethyl cellulose hydrolysates (see, e.g., Ichikawa, S.; Iwamoto, S.; Watanabe, J. Formation of biocompatible nanoparticles by self-assembly of enzymatic hydrolysates of chitosan and carboxymethyl cellulose. Biosci. Biotechnol. Biochem. 2005, 69, 1637-1642). The polymers were hydrolyzed with the enzymes chitosanase and cellulase, respectively. Electrostatic interactions between the carboxylate groups of carboxymethyl cellulose with the amino groups of chitosan resulted in spontaneous formation of nanoparticles just by mixing solutions of the two polymers. Particle size depended on the mixing ratio of the solutions, and also by the molecular weight of the polymers. It was necessary to hydrolyze the polymers and lower the molecular weight before mixing in order to prevent the formation of macroscopic gel.

Applicants synthesized hydroxypropyl cellulose microgels using relatively non-toxic crosslinking agents such as trisodium trimetaphosphate (TSTMP) and sodium tripolyphosphate (STPP). Hydroxypropyl cellulose (HPC) is prepared by base-catalyzed reaction of propylene oxide with cellulose. HPC is permitted in foods for human consumption, and is described under section 121.1160 of the U.S. Food and Drug Administration regulations [Klug, E. D. Hydroxypropyl Cellulose. In Encyclopedia of Polymer Science and Technology; Bikales, N. M., Ed.; Wiley Interscience: New York, 1971; Vol. 15, pp 307-314]. Up to 0.4 wt % of unreacted TSTMP and STPP are permissible in food products according to FDA regulations. Other reagents permitted by FDA for making food grade starch, such as phosphoryl chloride, adipate, and adipic-acetic mixed anhydride, may also be used for the crosslinking reaction. Carcinogens such as epichlorohydrin, although used in the past for crosslinking starch, can obviously not be used.

Crosslinking of starch using trisodium trimetaphosphate has been typically carried out in aqueous media at pH of 11.5 [Xie, S. X.; Liu, Q.; Cui, S. W. Starch modification and application. In Food Carbohydrates: Chemistry, Physical Properties, and Applications; Cui, S. W. Ed.; Taylor & Francis: New York 2005; p. 358]. The reaction is allowed to proceed at 40° C. for 2 to 6 h. The applicants found that hydroxypropyl cellulose microparticles could be obtained, at relatively high concentrations (up to 10 wt %, without macrophase separation), using significantly higher sodium hydroxide concentration and reaction temperature. Sodium hydroxide not only participates in the crosslinking reaction, but also, evidently, lowers the LCST of hydroxypropyl cellulose resulting in particle formation even at room temperature (at sufficiently high concentrations of NaOH).

Hydroxypropyl cellulose powder, obtained from Sigma-Aldrich, was used for microparticle synthesis. The HPC polymer had a number-average molecular weight, M̅ₙ, of 10,000 g/mol, a weight-average molecular weight, M̅_{w}, of 80,000 g/mol, a degree of substitution, DS, of 2.5, and a molar substitution, MS, of 3.7. The degree of substitution, DS, is defined as the average number of hydroxyl groups substituted per anhydroglucose unit [Klug, E. D. Hydroxypropyl Cellulose. In Encyclopedia of Polymer Science and Technology; Bikales, N. M., Ed.; Wiley Interscience: New York, 1971; Vol. 15, pp 307-314]. The molar substitution, MS, is defined as the average number of propylene oxide molecules combined per anhydroglucose unit.

About 15 mg of refined soy lecithin (MP Biomedicals) was dissolved in 5 mL of a sodium hydroxide solution (pH=12) to obtain a pale yellow translucent solution. Four hundred milligram of HPC was added to this solution and stirred to result in a viscous solution. In another vial, a 12% (w/v) solution of TSTMP was prepared in distilled water. Five milliliters of this TSTMP solution was then added to the HPC/soy lecithin solution. The mixture was stirred to obtain a homogeneous solution, which was heated at 50° C. for 1 h and subsequently cooled to room temperature. The pH of the resulting dispersion, measured using a stainless steel ISFET pH probe (IQ Scientific Instrument), was 7.8. The pH was adjusted to 7 using a few microliters of 4 M hydrochloric acid. The HPC dispersion consisted of: 400 mg of HPC (3.2 mmol of hydroxyl groups), 15 mg (0.05 mmol) soy lecithin, 600 mg (2.0 mmol) of TSTMP, and about 12 mg (0.3 mmol) sodium hydroxide in about 10 mL of distilled water. The number-average particle diameter was 3.5 μm and the weight-average particle diameter was 3.7 μm The viscosity of the dispersion was about 11 cP. Ten milliliters of a 20% (w/v) dextrose solution in distilled water was then added to this dispersion, and the mixture was heated at 60° C. for 10 min. The number-average particle diameter remained nearly the same (about 5 μm) after addition of dextrose. The viscosity of the final dispersion was about 5 cP. The average diameter of the particles in the dispersion was determined using a ALVS-NIBS High Performance Particle Sizer (ALV-GmbH, Langen/Germany). Dispersion viscosity was determined using a Ubbelohde Viscometer (Cannon Instrument Co., Pennsylvania).

There were no significant differences in the particle sizes or the dispersion viscosities when the formulations were heated at 50° C. for 3 h instead of 1 h.

In another formulation, 10 mL of a 4% (w/v) solution of HPC in distilled water was taken in a glass vial. Sodium hydroxide pellets (310 mg, 7.75 mmol) were added and dissolved in to this solution. The addition of sodium hydroxide resulted in a cloudy homogeneous dispersion. TSTMP (600 mg, 1.96 mmol) and soy lecithin (14 mg, 0.043 mmol) were subsequently added and dissolved. The dispersion was heated at 50° C. for 1 h, after which it was cooled to room temperature. The procedure resulted in the formation of macroparticles that settled to the bottom of the vial. Immediately after cooling, the dispersion was stirred (using a magnetic stirrer) and neutralized to pH 7 using 4 M hydrochloric acid. The number- and weight-average particle diameters in the supernatant phase were about 610 nm and 690 nm, respectively. The viscosity of the HPC dispersion was about 1.6 cP. Ten milliliters of a 20% (w/v) dextrose solution in distilled water was then added to this dispersion, and the mixture was heated at 60° C. for 10 min. The number-average particle diameter in the dextrose loaded dispersion was about 1.6 .mu.m and the weight-average particle diameter was about 2.2 μm after addition of dextrose. The viscosity of the final dispersion was about 2 cP.

In another embodiment, heating a solution of 4 g of HPC (31.9 mmol of hydroxyl groups) in 100 g of water with 2.1 g (52.5 mmol) of sodium hydroxide and 1 g (3.27 mmol) of TSTMP at 110° C. for 2 h, resulted in the formation of hydrogel microspheres. The dispersion was cooled to room temperature and neutralized using about 4 mL of 4 M hydrochloric acid to result in a solution with a viscosity of about 22 cP and a weight-average particle diameter of about 3.4 μm Addition of 104 mL of 20% (w/v) dextrose solution gave a final dispersion with a sugar concentration of 10% (w/v), a viscosity of 6.8 cP and a weight-average particle diameter of about 4.1 μm The formulation was heated at 60° C. for 10 min after the addition of sugar solution.

In another formulation, 8 g of HPC (63.7 mmol of hydroxyl groups) dissolved in 100 g of water was heated with 2.23 g (55.8 mmol) of sodium hydroxide and 1 g (3.27 mmol) of TSTMP. Heating was carried out in a sealed glass reactor at 110° C. for 2 h. After cooling, the unreacted sodium hydroxide was neutralized using about 20 mL of 4 M hydrochloric acid, to yield a dispersion of crosslinked HPC microspheres with a weight-average particle diameter of about 4.3 μm The viscosity of the dispersion was about 31.2 cP. A 20% (w/v) dextrose solution (120 mL) was then added to obtain a formulation with 10% (w/v) dextrose, 3.3% (w/v) HPC, about 2.5% (w/v) sodium chloride. The dispersion was heated at 60° C. for 10 min after sugar addition. The weight-average particle diameter in the final dispersion was about 4.5 μm, and the dispersion viscosity was about 31 cP. The dispersion viscosity was sensitive to the order in which the solutions were mixed. If the dextrose solution was added after the second heating step (60° C. for 10 min), the viscosity of the resulting dispersion was higher (about 55 cP).

Microparticle hydrogels of hydroxypropyl cellulose and sodium alginate (CAS no. 9005-38-3; American International Chemical, Inc., F-200) are synthesized as follows. Ten milligrams of HPC (0.080 mmol of hydroxyl groups) was dissolved in 1 mL of distilled water. To this solution was added 1 mL of 2.5 M NaOH solution (2.5 mmol NaOH), 20 mg (0.065 mmol) of trisodium trimetaphosphate, 10 mg of sodium alginate and 2 mg (6.1 μmol) of soy lecithin. The solution was stirred thoroughly. A cloudy dispersion was obtained that remained stable even after adding a few drops of concentrated hydrochloric acid (leading to a final pH of about 2, simulating the acidic environment of the stomach).

Hydroxypropyl cellulose self-assembles in water at a temperature greater than 41° C. This temperature, above which spontaneous self-assembly of the polymer chain occurs, is called the lower critical solution temperature (LCST). Methyl cellulose has an LCST between about 40° C. and 50° C. Hydroxypropyl methyl cellulose (HPMC) has been measured to have an LCST of about 73° C.

Thermal self-assembly of HPC, for example, is a reversible process. Individual polymer chains constituting the microparticles get solvated by water molecules when the solution is cooled below the LCST. Crosslinking the HPC chains using trisodium metaphosphate (TSTMP) prevents dissolution of the microparticles when the solutions are cooled below the critical solution temperatures.

In another strategy, crosslinking may be achieved by functionalizing the polysaccharide using acryloyl (or methacryloyl) groups using acryloyl chloride (or methacryloyl chloride). Formation of acryloyl esters results from the reaction of acryloyl chloride with the hydroxyl groups of the polysaccharide. It is important, however, to completely remove unreacted acryloyl chloride from the functionalized polymer, because of toxicity of acryloyl chloride. The vinyl functionalized HPC may then be crosslinked in water, above the LCST, using a relatively benign free-radical redox-initiator such as ascorbic acid and hydrogen peroxide, or thermal initiator such as potassium persulfate.

Thus, 1 g of hydroxypropyl cellulose (8 mmol) was taken in a round bottom flask equipped with a magnetic stir bar and fitted with a rubber septum. The polymer was dissolved in 20 mL of anhydrous dichloromethane to obtain a cloudy, viscous solution. The air in the flask was purged with dry nitrogen. About 1 mL (7 mmol) of triethyl amine was injected in to the reactor, followed by drop-wise addition of about 520 !IL (6.4 mmol) of acryloyl chloride. The mixture was stirred at room temperature, whereupon the cloudy solution became clear few minutes after the addition of acryloyl chloride. The solution was stirred overnight, after which the acrylated hydroxypropyl cellulose product was recovered and purified by repeated precipitations in cold (.about.0° C.) diethyl ether and acetone. The product was dried in vacuo at 40° C. About 40 mg of the acrylated HPC polymer was dissolved in 2 mL distilled water to obtain a cloudy solution at room temperature. About 65 mg (200 mmol) of soy lecithin was added to this solution and dissolved. The solution of HPC and soy lecithin was de-oxygenated by bubbling nitrogen gas, after which a 2 mL of a degassed solution of ammonium persulfate (9.1 mg, 40 mmol) was injected. The solution was heated at 70° C. for 2 h to obtain a dispersion of crosslinked acrylated hydroxypropyl cellulose particles. The number-average and weight-average particle diameters were 1.28 μm and 1.34 μm, respectively.

In an emulsion-based synthesis of hydroxypropyl cellulose microgels, 80 mg of acrylated hydroxypropyl cellulose was dissolved in 2 mL of dichloromethane. Distilled water (4 mL) was added to this solution and stirred to obtain an emulsion. Crosslinking of the acrylated hydroxypropyl cellulose was carried out at 35° C. using a redox system of ammonium persulfate and dextrose. Dextrose (21.6 mg, 12 mmol) was dissolved in the emulsion. Two milliliters of a solution of ammonium persulfate (27.4 mg, 0.12 mmol) in distilled water (2 mL) was injected in to the emulsion to initiate the crosslinking reaction. Dichloromethane was removed from the resulting dispersion using a rotary evaporator. A cloudy dispersion of crosslinked acrylated hydroxypropyl cellulose microgels was obtained. The crosslinked particles settled to the bottom of the vial on standing, and could therefore be isolated in a powder form by decanting the supernatant. The crosslinking may also be carried out using redox systems such as persulfate/glucose, hydrogen peroxide/ascorbic acid, etc.

Scanning electron microscopy of a 400 mg HPC, 100 mg TSTMP, 200 mg NaOH, 10 mL water solution heated at 110° C. for 2 h, wherein the dispersion was neutralized with concentrated HCl acid revealed large (about 1 nm) cubic particles seen under SEM. HPC has a low glass transition temperature and readily forms a film on the SEM substrate at room temperature. However, it was difficult to image the nanoparticles using SEM.

Other studies have shown that the rate of exogenous CHO oxidation can be increased by using a mixture of different monosaccharides (e.g., glucose, fructose, and sucrose). Jentjens et al. found that when glucose was ingested at a rate of 1.8 g glucose per minute, the rate of exogenous CHO oxidation was limited to 0.83 g/min. On the other hand, when a mixture of glucose and fructose was ingested, a total exogenous CHO oxidation rate of 1.26 g/min could be achieved—a 52% increase. An earlier study by Adopo et al. had shown that ingestion of a mixture of glucose and fructose resulted in higher exogenous CHO oxidation rates than an isocaloric amount of glucose. The oxidation rate of the exogenous glucose and fructose was 21% higher than the rate when only glucose was consumed. Because different monosaccharides are transported across the intestinal lumen by specific transport proteins, a mixture of monosaccharides may result in a higher overall uptake by cells than a single carbohydrate. For example, while glucose and galactose are transported through intestinal cell membranes by a transport protein called sodium-dependent glucose transporter 1 (SGLT1), fructose is transported by a different transport protein called glucose transporter 5 (GLUT5). In principle, supplying a 1:1 mixture of glucose and fructose molecules will reduce traffic in the SGLT1 transport pathway by a factor of 2, compared to the case where only glucose molecules are provided. Although the net rate of absorption of CHOs may increase using a mixture of glucose and fructose, fructose may not be immediately available as energy source, because of the relatively slow rate of hepatic conversion of fructose to glucose.

The blood flow rate to the small intestine could also be a limiting factor in CHO absorption. There is a significant decrease in the blood flow rate to small intestine during high intensity exercise. The reason for a limiting exogenous glucose oxidation rate during exercise could also be due to reduced blood flow rate to small intestine. It is also likely that hepatic glycogen synthesis and glycogenolysis do not allow a glucose output greater than about 1.0 g/min, regardless of the supply rate from the small intestine.

Microparticles of a temperature responsive polymer, such as hydroxypropyl cellulose (HPC), were prepared by heating an aqueous solution of the polymer above its lower critical solution temperature. The polymer chains within the particles were covalently crosslinked using FDA-approved trisodium trimetaphosphate (TSTMP), to obtain microparticle hydrogels. The particles were loaded with dextrose (D-glucose) and the rates of release of entrapped dextrose were studied for formulations with different chemical compositions and particle concentrations. The sugar that was present within the water-swollen hydrogel particles were available for delayed release. The remaining sugar was present in the aqueous phase, and was available for immediate absorption across the intestinal lumen. The hydrogel microparticles comprised a pH responsive, mucoadhesive polymer, such as sodium alginate, to provide a diffusional barrier against gastric release. Both in vitro release kinetics and in vivo release kinetics (at two different rates of energy expenditure) were experimentally determined. Glucose concentration versus time profiles for delayed-release formulations suitable for use in the methods of the present invention showed clear differences and advantages over conventional immediate release formulations available in the market, and other controls. See, for example, U.S. Patent Application Publication No. 2012/0015039, incorporated herein by reference.

### Materials

Hydroxypropoyl cellulose (HPC-SL, USP grade) was received from Nippon Soda Co. Ltd. Refined soy lecithin was purchased from MP Biomedicals Inc., LLC (catalog no. 102147). Sodium alginate polymers (sodium alginate NF, F-200, SAHMUP and sodium alginate NF, SALMUP) were received from American International Chemical, Inc. Trisodium trimetaphosphate (TSTMP, reagent grade) and sodium hydroxide (reagent grade, >98%) were purchased from Sigma-Aldrich. The glucose oxidase/peroxidase enzymes (PGO enzymes capsules, product no. P7119), o-dianisidine dihydrochloride (catalog no. D3252), dextrose (catalog no. D9434) and hydrochloric acid (37%, catalog no. 320331) were obtained from Sigma-Aldrich. Thin-N-Thik® 99 starch and Resista® 682 starch, anhydrous citric acid, Staleydex® 333 dextrose, and Krystar®. 300 crystalline fructose, were received from Tate & Lyle. Food grade soy lecithin, UltraLec®. P Deoiled Lecithin was received from Archer Daniels Midland Company. The food grade surfactant, diacetyl tartaric acid ester of monoglyceride (DATEM, Panodan® 150 LP K-A) was received from Danisco. Sodium hydroxide (FCC grade) was purchased from VWR. Sodium benzoate (FCC grade) was purchased from Fischer Scientific. Food grade potassium sorbate and trisodium trimetaphosphate were purchased from Spectrum Chemical Mfg. Corp. All the chemicals were used without further purification. A widely used commercial sports drink, GATORADE®, was used as a positive control for the in vivo experiments. GATORADE® consists of water, high fructose corn syrup (glucose-fructose syrup), sucrose syrup, citric acid, natural flavor, salt, sodium citrate, monopotassium phosphate, modified food starch, red dye # 40, and glycerol ester of rosin. The total sugar concentration is 5.83% (w/v). The sodium and potassium concentrations are 0.45 mg/mL and 0.125 mg/mL, respectively.

### Hydroxypropyl Cellulose (HPC)

Hydroxypropyl cellulose is a temperature-responsive polymer. When heated above the lower critical solution temperature (LCST) of the polymer solution, the hydrated polymer chains lose water because of thermal disruption of polymer-water hydrogen bonds. The polymer chains precipitate out of solution, as they become hydrophobic, to form microparticles. Particle formation by hydrophobic interaction is reversible—the polymer molecules become soluble again when the dispersion is cooled below the LCST. The effect of different additives on the lower critical solution temperature of an aqueous HPC solution was determined using Differential Scanning calorimetry. The LCST of an aqueous solution of HPC (8% w/v) was 48° C. When 4 mL of 3.2% (w/v) soy lecithin solution was added to an 8% (w/v) HPC solution (10 mL), no change in the LCST was observed. When 3 g of TSTMP solution in water (1.77% w/v) was added to the solution containing HPC and soy lecithin, the LCST decreased to 37° C. Finally, 0.5 g of a 1.36% w/v sodium hydroxide solution was added and the dispersion was heated for 1 h at 50° C., with stirring at 300 rpm. A solid precipitate of polymer particles was observed after 1 h of heating, which could be easily re-dispersed after cooling to room temperature. The pH of the dispersion was adjusted to about 7 by adding 40 .mu.L of 4 N hydrochloric acid. Dextrose (1.75 g) was added to the dispersion and was dissolved by stirring. The LCST of the crosslinked HPC in dispersion, after addition of dextrose, was about 32° C. From these measurements of the effect of additives on the LCST of HPC, it is evident that particle formation occurs even without the use of a crosslinker. Chemical crosslinking is, however, desirable to maintain particle integrity over a wider range of ionic strength, temperature and pH conditions.

The degree of substitution (DS) and molar substitution (MS) are important parameters that affect particle foimation and crosslinking in HPC dispersions. Each glucose unit in the cellulose molecule has three hydroxyl groups. The degree of substitution is defined as the average number of hydroxyl groups per anhdryoglucose unit that have reacted with the propylene oxide. Therefore, the degree of substitution is always less than or equal to three. Molar substitution is defined as the average number of propylene oxide molecules that have reacted per glucose unit. The molar substitution is generally greater than the degree of substitution, and can be greater than 3. The ratio of molar substitution to degree of substitution gives the average length of the hydroxypropyl side chains in the polymer.

Based on the structure of the HPC polymer, it is evident that the average molecular weight of each repeat unit in the polymer is equal to (162.15+58.08 MS). Each repeat unit has three hydroxyl groups. Hence, the number of moles of hydroxyl group per gram of the HPC polymer is given by 3/(162.15+58.08 MS). For HPC-SL, the degree of substitution is 1.9, and the molar substitution is about 2.1. Hence, the concentration of hydroxyl groups is about 10.6 mmol per gram of the polymer.

### Dispersion Synthesis

At the reaction temperature of 50° C., the HPC chains aggregated to form microparticles. The individual polymer chains in the particles were covalently crosslinked. At the end of the crosslinking reaction, the particles settled at the bottom of the vial. They could, however, be easily re-dispersed by gentle stirring, after cooling to the room temperature.

An IQ150-77 pH/mV/Temperature system (IQ Scientific Instruments) with a general purpose stainless steel ISFET sensor probe was used for pH measurements. Particle sizes in the dispersions were measured using ALV-NIBS High Performance Particle sizer. Scanning electron microscopy was done using a JEOL JSM 6300 scanning electron microscope. A drop of the sample was air dried on an aluminum stub for about 12 hours at room temperature. The dry particles were sputter coated with a conducting layer of gold before the SEM analysis. The viscosities of the dispersions were determined using an Ubbelohde viscometer (Cannon instruments Co., size 1C). The time taken for the liquid to elute between two fiducial points on the viscometer was measured using a stopwatch, and the viscosity of the formulation was calculated as the product of the ‘viscometer constant’, the experimentally determined liquid density, and the elution time. Differential scanning calorimetry (DSC) was performed using a TA Instruments Differential Scanning calorimeter. The DSC measurements were made in an inert atmosphere of ultra high purity nitrogen. PerkinElmer aluminum pans (# 02190062) were used for both the sample and the reference. The samples were heated to 75° C., held at this temperature for 1 minute, and then cooled to 20° C. at a rate of 10° C./min. The difference in heat flow between the sample and reference was measured to obtain the DSC thermogram.

In vitro release kinetics, of glucose encapsulated in the hydrogel microparticles, was determined using PermeGear Side-Bi-Side horizontal diffusion cell. The diffusion cell consisted of a donor and receiver chamber separated by a membrane. The membrane was placed between the two chambers and the chambers were held together with a stainless steel clamp. The donor and receiver chamber had a volume of 7 mL each, and the diameter of the orifice was 15 mm. Both the donor and receiver chamber were surrounded by jackets through which water from a temperature controlled water bath was circulated. For release kinetics experiments, a polyethersulfone membrane was used because of its hydrophilicity and acid resistance. Polyethersulfone membranes with 450 nm pore size, and 25 mm diameter were purchased from Sterlitech Corporation. The diffusion cell assembly was mounted on a magnetic stir plate. The contents of the receiver chamber were stirred using a magnetic stir bar. The contents of the donor chamber were left unstirred. For the determination of glucose concentration as a function of time, 100-μL samples were withdrawn from the receiver chamber using a microsyringe, and replaced with an equal volume of distilled water.

Glucose concentrations in the in vitro experiments were determined using a colorimetric glucose oxidase method, following a Sigma-Aldrich protocol. The glucose oxidase/peroxidase enzyme solution was prepared by dissolving 1 capsule of Sigma's PGO Enzymes in 100 mL of water in an amber bottle. Each capsule contained 500 units of glucose oxidase (Aspergilus niger), 100 purpurogallin units of peroxidase (horseradish), and buffer salts. The bottle was inverted several times with gentle shaking to dissolve the capsule. The o-dianisidine solution was prepared by dissolving 50 mg of o-dianisidine dihydrochloride in 20 mL of water. The PGO-enzymes reaction solution was prepared by mixing 100 mL of the PGO enzyme solution and 1.6 mL of the o-dianisidine dihydrochloride solution. The solution was mixed by inverting several times or with mild shaking. A glucose standard of 0.05 mg/ml in water was prepared. The glucose-containing sample was added to the PGO enzymes reaction solution. The reaction was allowed to proceed to completion in approximately 45 minutes at room temperature. The final absorbance was measured using a PerkinElmer Lambda 650 UV-vis spectrophotometer at 450 nm wavelength. The glucose concentration of the sample was determined as follows:

$$Sample Glucose Concentration ( mg / mL ) = \frac{Absorbance ( Test ) \times Dilution of Sample \times 0.05 mg / mL}{Absorbance ( Standard )}$$

Glucose is oxidized to gluconic acid and hydrogen peroxide by glucose oxidase. Hydrogen peroxide reacts with o-dianisidine in the presence of peroxidase to form a colored product. The intensity of the brown color measured at 450 nm is proportional to the original glucose concentration.

The invention will now be further described by way of the following non-limiting examples.

### EXAMPLES

### Preparation of HPC Microgel Particles Containing Metformin

Crosslinked hydroxypropyl cellulose (HPC) microparticle dispersions were synthesized for sustained release of metformin hydrochloride. HPC was dissolved in water to obtain a 9 wt % solution. A surfactant dispersion was prepared by adding 2.66 g of diacetyl tartaric acid ester of mono- and diglycerides (DATEM) to 604.80 g of hot water. Upon cooling to room temperature, a colloidal dispersion of DATEM in water was obtained. To 362 g of the DATEM dispersion, about 3.5 g of sodium hydroxide was added. A solution of the crosslinker was prepared by dissolving 52.86 g trisodium trimetaphosphate (TSTMP) in 283.34 g water. Next, 366 g of DATEM dispersion (containing sodium hydroxide) was added to the HPC solution with mixing using an overhead stirrer. 296 g of the TSTMP solution was added to this mixture. The temperature of the mixture was then increased to 50° C. The reaction was allowed to proceed for two hours with mixing. After that, agitation was ceased and the crosslinked particles were allowed to settle to the bottom of the reactor. The supernatant solution was removed and the particles were rinsed with hot water. After rinsing, the particles were reconstituted and the reactor was cooled to room temperature. The pH was decreased to about 7 using 5 N hydrochloric acid. An aliquot of the particle suspension was then removed and metformin hydrochloride was added to achieve a 100 mg/mL suspension.

### Metformin Release Kinetics

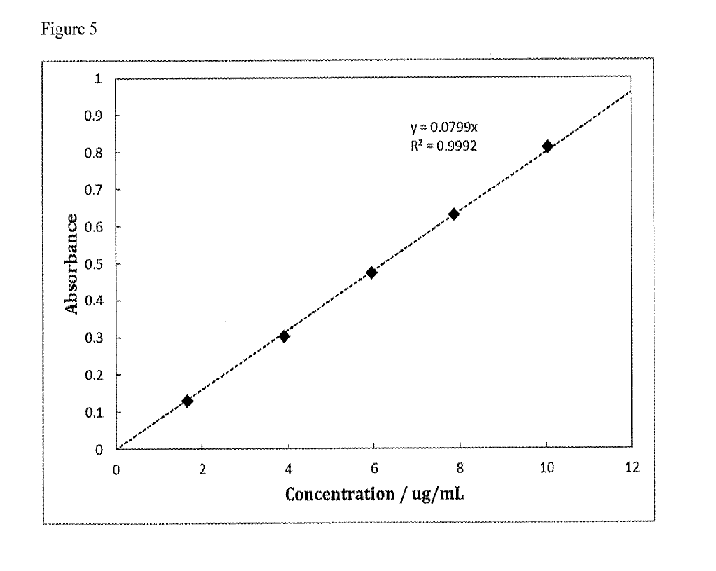

Metformin can be quantified using UV-Vis spectroscopy. FIG. 5 depicts a metformin hydrochloride (MH) calibration curve, demonstrating the ability of metformin to be readily quantified using UV-Vis spectroscopy.

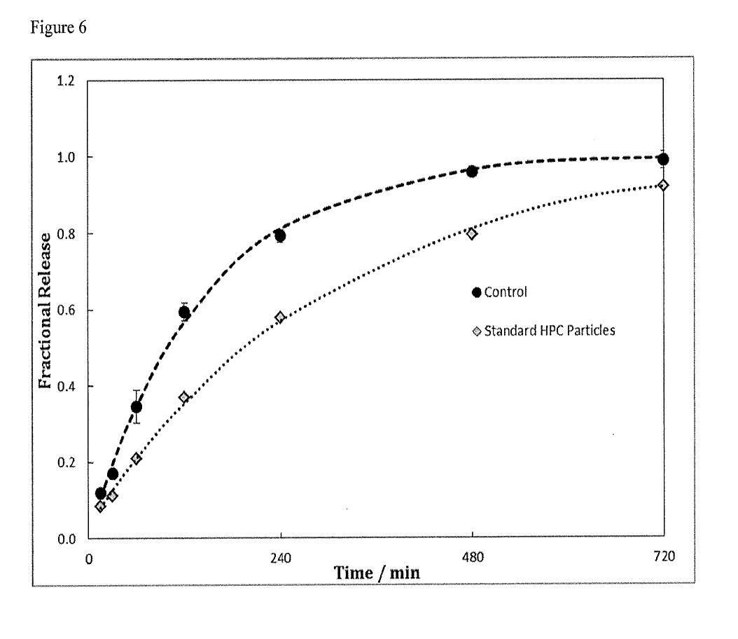

The release kinetics of metformin from the HPC microgel particles prepared as above was investigated. FIG. 6 depicts the release kinetics of metformin hydrochloride (MH) using a horizontal static diffusion cell. The control experiments were performed with 100 mg/mL MH solution at 37° C. with phosphate buffered saline (PBS) as the receptor medium. The standard HPC particle suspension saturated with MH provides a delay in the release of MH over an eight hour period. In further embodiments, functionalizing the standard particles with negatively charged carboxymethyl cellulose (CMC) or alginate should provide a large separation in the release kinetics profiles between control and formulation.

Preparation of HPC-CO-CMC Particles Via Temperature Responsive Crosslinking

The chemical crosslinking of hydroxypropyl cellulose (HPC) with carboxymethyl cellulose (CMC) was attempted with trisodium trimetaphosphate (TSTMP). Briefly, a surfactant dispersion was prepared by adding 500 mg of diacetyl tartaric acid ester of mono- and diglycerides (DATEM) in 500 mL of water at 80° C. This mixture was stirred while cooling to room temperature. Once at room temperature, 50 g of HPC-L was added to the mixture. After one hour of mixing the HPC had dissolved. A 100 g aliquot was removed for small scale experiments. To the remaining mixture, 4 g of CMC was added. The mixture was stirred for one hour to dissolve the CMC. With continued agitation, 50 g of TSTMP and 100 g of deionized water were added to the reactor. The mixture was stirred for ten minutes before the pH was elevated to 12.4 with about 18 g of 2 M NaOH. The reactor was then raised to 50° C. and the particle reaction was allowed to proceed for three hours. Agitation was ceased after three hours. The formed solids did not rapidly settle out of suspension. Approximately 500 g of 50° C. deionized water was added to the mixture to lower the pH and density of the aqueous phase. The mixture was again stirred for ten minutes before agitation was stopped. Again the particles did not readily settle out of suspension. In order to precipitate any uncrosslinked CMC, 5 N HCl was added to the mixture to reduce the pH below the pKa of CMC (˜2.8). The mixture changed visibly upon decreasing the pH to about 2.3 and the solids quickly settled to the bottom of the reactor. The supernatant was removed and replaced with approximately 500 g of 50° C. deionized water. The reactor contents were again mixed for ten minutes to rinse the particles. Agitation was stopped and the particles again quickly settled to the bottom. The rinse supernatant was removed and the rinse process was repeated a second time in its entirety. Finally, the particles were reconstituted to approximately the original mixture volume and the reactor was reduced to room temperature to rehydrate the particles. After reaching room temperature, the system pH was 2.77. The suspension pH was elevated to 7.4 with about 5 g of 2 M NaOH.

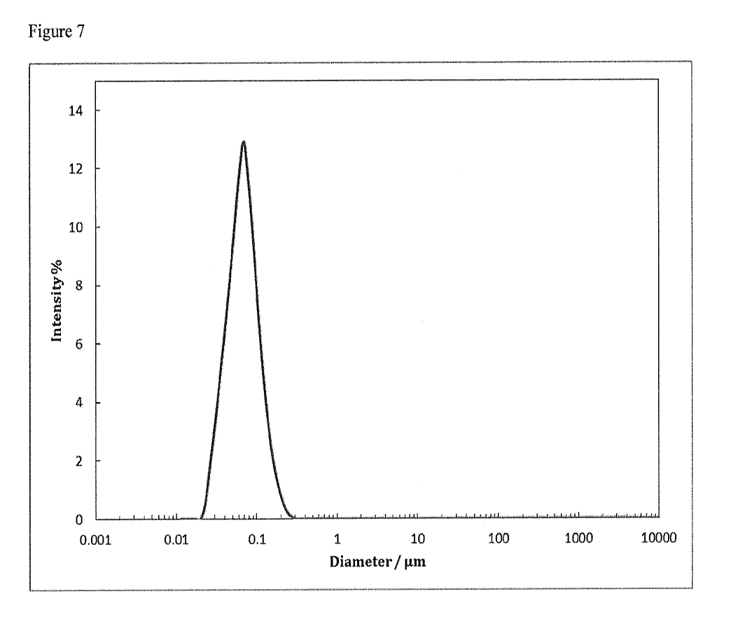

The particle formation process relies on the temperature induced aggregation of HPC above its lower critical solution temperature (LCST). Above this temperature, the HPC chains lose solubility. FIG. 7 depicts laser diffraction analysis of particles formed by the temperature-induced precipitation crosslinking of HPC and CMC with TSTMP as described above. Without being bound to theory, the significantly smaller particle sizes obtained by this method suggest that CMC, which is hydrophilic at the elevated pH of the crosslinking step, acts as a surfactant reducing the hydrophobicity of the HPC at temperatures above its lower critical solution temperature (LCST).

### REFERENCES

1. Jeukendrup A, Brouns F, Wagenmakers A J, Saris W H. Carbohydrate-electrolyte feedings improve 1 h time trial cycling performance. Int j Sports Med. 1997 February; 18 (2):125-9.
2. Coyle E F, Coggan A R, Hemmert M K, Ivy J L. Muscle glycogen utilization during prolonged strenuous exercise when fed carbohydrate. J Appl Physiol. 1986 July; 61 (1):165-72.
3. Bergstrom J, Hermansen, L., Hultman, E., Saltin, B. Diet, muscle glycogen and physical performance. Acta Physiol Scand. 1967; 71 (2):140-50.
4. Costill D L. Carbohydrate for athletic training and performance. Bol Asoc Med P R. 1991 August; 83 (8):350-3.
5. S. Samols E, Dormandy, T. L. Insulin response to fructose and galactose. Lancet. 1963; 1 (7279):478-9.
6. Jandrain B J, Pallikarakis N, Normand S, Pirnay F, Lacroix M, Mosora F, et al. Fructose utilization during exercise in men: rapid conversion of ingested fructose to circulating glucose. J Appl Physiol. 1993 May; 74 (5):2146-54.
7. Chen M, Whistler R L. Metabolism of D-fructose. Adv Carbohydr Chem Biochem. 1977; 34:265-343.
8. Leijssen D P, Saris W H, Jeukendrup A E, Wagenmakers A J. Oxidation of exogenous [13C]galactose and [13C]glucose during exercise. J Appl Physiol. 1995 September; 79 (3):720-5.
9. Hawley J A, Dennis S C, Nowitz A, Brouns F, Noakes T D. Exogenous carbohydrate oxidation from maltose and glucose ingested during prolonged exercise. Eur J Appl Physiol Occup Physiol. 1992; 64 (6):523-7.
10. Rehrer N J, Wagenmakers A J, Beckers E J, Halliday D, Leiper J B, Brouns F, et al. Gastric emptying, absorption, and carbohydrate oxidation during prolonged exercise. J Appl Physiol. 1992 February; 72 (2):468-75.
11. Little T J, Doran S, Meyer J H, Smout A J, O'Donovan D G, Wu K L, et al. The release of GLP-1 and ghrelin, but not GIP and CCK, by glucose is dependent upon the length of small intestine exposed. Am J Physiol Endocrinol Metab. 2006 September; 291 (3):E647-55.
12. Austin J, Marks D. Hormonal regulators of appetite. International journal of pediatric endocrinology. 2009; 2009:141753.
13. Parks D A, Jacobson E D. Physiology of the splanchnic circulation. Arch Intern Med. 1985 July; 145 (7):1278-81.
14. Perko M J, Nielsen H B, Skak C, Clemmesen J O, Schroeder T V, Secher N H. Mesenteric, coeliac and splanchnic blood flow in humans during exercise. J Physiol. 1998 Dec. 15; 513 (Pt 3):907-13.
15. Physiology of Sport and Exercise. 2nd ed. Champaign, IL: Human Kinetics; 1999. 710 p.
16. Exercise physiology: human bioenergetics and its application. 3rd ed. Mountain View, Calif.: Mayfield Publishing C.; 2000.
17. Dudley G A, Abraham W M, Terjung R L. Influence of exercise intensity and duration on biochemical adaptations in skeletal muscle. J Appl Physiol. 1982 October; 53 (4):844-50.
18. Haff G G. Carbohydrates. In: Antonio J, Kalman, D., Stout, J. R., Greenwood, M., Willoughby, D. S., Haff, G. G., editor. Essentials of Sports Nutrition and Exercise: Humana Press; 2008. p. 281-311.
19. Pitsiladis Y P, Maughan R J. The effects of exercise and diet manipulation on the capacity to perform prolonged exercise in the heat and in the cold in trained humans. J Physiol. 1999 Jun. 15; 517 (Pt 3):919-30.
20. Pitsiladis Y P, Maughan R J. The effects of alterations in dietary carbohydrate intake on the performance of high-intensity exercise in trained individuals. Eur J Appl Physiol Occup Physiol. 1999 April; 79 (5):433-42.
21. Vandenbogaerde T J, Hopkins, W. G. Effects of acute carbohydrate supplementation on endurance performance: a meta-analysis. Sports Med. 2011; 41 (9):773-92.
22. Newsholme E A, Blomstrand E, Ekblom B. Physical and mental fatigue: metabolic mechanisms and importance of plasma amino acids. Br Med Bull. 1992 July; 48 (3):477-95.
23. Matsui T, Soya S, Okamoto M, Ichitani Y, Kawanaka K, Soya H. Brain glycogen decreases during prolonged exercise. J Physiol. 2011 Jul. 1; 589 (Pt 13):3383-93.
24. Matsui T, Ishikawa T, Ito H, Okamoto M, Inoue K, Lee M C, et al. Brain glycogen supercompensation following exhaustive exercise. J Physiol. 2012 Feb. 1; 590 (Pt 3):607-16.
25. Peters A, Schweiger U, Pellerin L, Hubold C, Oltmanns K M, Conrad M, et al. The selfish brain: competition for energy resources. Neurosci Biobehav Rev. 2004 April; 28 (2):143-80.
26. Goodman & Gilman's The Pharmacological Basis of Therapeutics. 11th ed. New York, N.Y.: McGraw-Hill; 2006.
27. Davis J K, Green J M. Caffeine and anaerobic performance: ergogenic value and mechanisms of action. Sports Med. 2009; 39 (10):813-32.
28. Yeo S E, Jentjens R L, Wallis G A, Jeukendrup A E. Caffeine increases exogenous carbohydrate oxidation during exercise. J Appl Physiol. 2005 September; 99 (3):844-50.
29. Burke L M. Caffeine and sports performance. Appl Physiol Nutr Metab. 2008 December; 33 (6):1319-34.
30. Ivy J L, Kammer L, Ding Z, Wang B, Bernard J R, Liao Y H, et al. Improved cycling time-trial performance after ingestion of a caffeine energy drink. Int J Sport Nutr Exerc Metab. 2009 February; 19 (1):61-78.
31. Cox G R, Desbrow B, Montgomery P G, Anderson M E, Bruce C R, Macrides T A, et al. Effect of different protocols of caffeine intake on metabolism and endurance performance. J Appl Physiol. 2002 September; 93 (3):990-9.
32. Bridge C A, Jones M A. The effect of caffeine ingestion on 8 km run performance in a field setting. J Sports Sci. 2006 April; 24 (4):433-9.
33. Astorino T A, Matera A J, Basinger J, Evans M, Schurman T, Marquez R. Effects of red bull energy drink on repeated sprint performance in women athletes. Amino Acids. 2012 May; 42 (5):1803-8.
34. Hendrix C R, Housh T J, Mielke M, Zuniga J M, Camic C L, Johnson G O, et al. Acute effects of a caffeine-containing supplement on bench press and leg extension strength and time to exhaustion during cycle ergometry. J Strength Cond Res. 2010 March; 24 (3):859-65.
35. Hunter A M, St Clair Gibson A, Collins M, Lambert M, Noakes T D. Caffeine ingestion does not alter performance during a 100-km cycling time-trial performance. Int J Sport Nutr Exerc Metab. 2002 December; 12 (4):438-52.
36. Hogervorst E, Riedel W J, Kovacs E, Brouns F, Jolles J. Caffeine improves cognitive performance after strenuous physical exercise. Int J Sports Med. 1999 August; 20 (6):354-61.
37. Clauson K A, Shields, K. M., McQueen, C. E., Persad, N. Safety issues associated with commercially available energy drinks. J Am Pharm Assoc. 2008; 48:e55-e63.
38. Graham T E, Spriet, L. L. Metabolic, catecholamine, and exercise performance responses to various doses of caffeine. J Appl Physiol. 1995; 78:867-74.
39. Ballard S L, Wellborn-Kim, J. J., Clauson, K. A. Effects of commercial energy drink consumption on athletic performance and body composition. Phys Sportsmed. 2010; 38:107-17.
40. Armstrong L E. Caffeine, body fluid-electrolyte balance and exercise performance. Int J Sport Nutr Exerc Metab. 2002; 12:189-206.
41. Millard-Stafford M L, Cureton, K. J., Wingo, J. E., Trilk, J., Warren, G. L., Buyckx, M. Hydration during exercise in warm, humid conditions: effect of caffeinated sports drink. Int J Sport Nutr Exerc Metab. 2007; 17:163-77.
42. Wemple R D, Lamb, D. R., McKeever, K. H. Caffeine vs. caffeine-free sports drinks: effects on urine production at rest and during prolonged exercise. Int J Sports Med. 1997; 18:40-6.

Having thus described in detail embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

What is claimed is:

1. A method of improving cognitive function, comprising administering to a subject in need thereof a composition comprising one or more hydrogel particles, wherein the one or more hydrogel particles
(a) are non-toxic; and
(b) incorporate at least one active agent, wherein the one or more hydrogel particles release the active agent in a time-controlled and sustained manner in vivo,
wherein the administration of the composition improves cognitive function in the subject.

2. A method of treating a central nervous system (CNS) disease or condition, comprising administering to a subject in need thereof a composition comprising one or more hydrogel particles, wherein the one or more hydrogel particles
(a) are non-toxic; and
(b) incorporate at least one active agent, wherein the one or more hydrogel particles release the active agent in a time-controlled and sustained manner in vivo,
wherein the administration of the composition improves brain and/or spinal cord function in the subject.

3. The method of claim 2, wherein the CNS disease or condition is selected from the group consisting of: ischemia, a neurodegenerative disorder, a mental health disorder, a pain disorder, an addiction disorder, a brain or spinal cord injury, and a brain or spinal cord tumor.

4. A method of treating a metabolic disorder, comprising administering to a subject in need thereof a composition comprising one or more hydrogel particles, wherein the one or more hydrogel particles
(a) are non-toxic; and
(b) incorporate at least one active agent, wherein the one or more hydrogel particles release the active agent in a time-controlled and sustained manner in vivo,
wherein the administration of the composition improves metabolic function in the subject.

5. The method of claim 4, wherein the metabolic disorder is selected from the group consisting of: obesity, metabolic syndrome, and hypoglycemia.

6. The method of claim 4, wherein the metabolic disorder is selected from the group consisting of diabetes, insulin resistance, hyperglycemia, and impaired glucose tolerance.

7. A method of increasing satiety hormone release, comprising administering to a subject in need thereof a composition comprising one or more hydrogel particles, wherein the one or more hydrogel particles
(a) are non-toxic; and
(b) incorporate at least one active agent, wherein the one or more hydrogel particles release the active agent in a time-controlled and sustained manner in vivo,
wherein the administration of the composition increases satiety hormone release in the subject.

8. The method of claim 7, wherein the satiety hormone is selected from cholecystokinin (CCK), peptide YY (PYY), pancreatic polypeptide (PP), insulin, and incretins.

9. The method of claim 8, wherein the incretin is selected from the group consisting of: glucagon-like peptide 1 (GLP-1), oxyntomodulin, and glucose-dependent insulinotropic polypeptide.

10. A method of decreasing hunger hormone release, comprising administering to a subject in need thereof a composition comprising one or more hydrogel particles, wherein the one or more hydrogel particles
(a) are non-toxic; and
(b) incorporate at least one active agent, wherein the one or more hydrogel particles release the active agent in a time-controlled and sustained manner in vivo,
wherein the administration of the composition decreases hunger hormone release in the subject.

11. The method of claim 10, wherein the hunger hormone is ghrelin.

12. The method of claim 1, 2, 4, 7, 10, or 32 wherein the at least one active agent is a carbohydrate.

13. The method of claim 12, wherein the carbohydrate is selected from the group consisting of: monosaccharides, disaccharides, polysaccharides, and combinations thereof.

14. The method of claim 13, wherein the carbohydrate is selected from the group consisting of: glucose, fructose, galactose, sucrose, maltose, lactose, dextrose, polydextrose, dextrins, maltodextrins, corn syrup solids, starch, and combinations thereof.

15. The method of claim 14, wherein the carbohydrate is glucose.

16. The method of claim 15, wherein the glucose is released in distal portions of the small intestine after administration of the composition to the subject.

17. The method of claim 1 or 2, wherein the active agent improves neurotransmitter efficacy.

18. The method of claim 1, wherein the active agent increases brain glycogen stores.

19. The method of claim 1, wherein improvements in cognitive function include improvements in attention, psychomotor, and/or memory abilities.

20. The method of claim 1, 2, 4, 7, 10, or 32, wherein the one or more hydrogel particles comprise one or more compounds that are temperature-sensitive.

21. The method of claim 20, wherein the one or more compounds have a lower critical solution temperature in aqueous solution.

22. The method of claim 1, 2, 4, 7, 10, or 32, wherein the one or more hydrogel particles comprise one or more compounds that are pH-sensitive.

23. The method of claim 22, wherein the one or more compounds do not swell at pH 1-3.

24. The method of claim 20, wherein the one or more hydrogel particles further comprise one or more compounds that are pH-sensitive.

25. The method of claim 24, wherein the one or more compounds do not swell at pH 1-3.

26. The method of claim 1, 2, 4, 7, 10, or 32, wherein the one or more hydrogel particles comprise one or more compounds that are crosslinked.

27. The method of claim 1, 2, 4, 7, 10, or 32, wherein the one or more hydrogel particles have a diameter between about 1 nanometer to about 1000 micrometers.

28. The method of claim 4, wherein the active agent is metformin.

29. The method of claim 6, wherein the metabolic disorder is diabetes and the diabetes is type 2 diabetes.

30. The method of claim 29, wherein the active agent is metformin.

31. The method of claim 2, wherein the active agent is levodopa or phenylalanine.

32. A method of treating a cardiovascular disorder, a digestive disorder, an immune disorder, a pulmonary disorder, a viral disease, or a cancer, comprising administering to a subject in need thereof a composition comprising one or more hydrogel particles, wherein the one or more hydrogel particles
(a) are non-toxic; and
(b) incorporate at least one active agent, wherein the one or more hydrogel particles release the active agent in a time-controlled and sustained manner in vivo,
wherein the administration of the composition improves the cardiovascular, digestive, immune, and/or pulmonary function in the subject and/or treats the viral disease and/or cancer in the subject.

33. The method of claim 32, wherein the active agent is selected from the group consisting of: pravastatin, cimetidine, methotrexate, theophylline, and zidovudine.

34. The method of claim 1, 2, 4, 7, 10, or 32, wherein the bioavailability of the active agent is increased.
